(19) United States
(12) Patent Application Publication
(10) Pub. No.: US 2006/0276440 A1
(43) Pub. Date: Dec. 7, 2006

(54) Treatment of inflammatory disorders

(76) Inventors: Wenqian Frank An, Framingham, MA (US); Jun Young Park, Brookline, MA (US); Mehran Khodadoust, Brookline, MA (US); Hans-Jurgen Hess, Olde Lyme, CT (US); Md. Sajjat Hussom, Lexington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/325,615

(22) Filed: Jan. 3, 2006

Related U.S. Application Data
(60) Provisional application No. 60/641,041, filed on Jan. 3, 2005.

(51) Int. Cl. A61K 31/57; A61K 31/517; A61K 31/428; A61K 31/366; A61K 31/203

(52) U.S. Cl. 514/169; 514/367; 514/394; 514/464; 514/469; 514/266.24; 514/457; 514/559; 514/625

(57) ABSTRACT

Methods of treating an inflammatory disorder include administering an effective amount of a compound represented by Structural Formula (I): 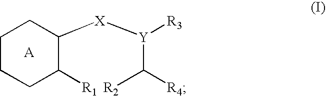
Ring A is optionally substituted, contains zero, one, two, or three double bonds, and is optionally fused to an aliphatic, aryl or heteroaryl ring; X is an optionally substituted 1 to 3 carbon aliphatic chain that is optionally fused to a monocyclic, optionally substituted, aliphatic, heterocyclic, aryl, or heteroaryl ring, wherein one or two carbons in X are optionally replaced with —O—, —S—, or —NR^{e}—; Y is carbon or nitrogen; R₁ and R₂ are independently —H, —OH, —CN, —NO₂, —NR^{f}R^{g}, halogen, optionally substituted alkyl, or optionally substituted alkoxy; or R₁ and R₂ together link the carbons to which they are bonded with a bond, —O—, —S—, or —NR^{h}—; R₃ and R₄ are independently —H, —OH, —CN, —NO₂, —NRⁱR^{j}, halogen, optionally substituted alkyl, or optionally substituted alkoxy, or R₄ is ═O; or R₃ and R₄, taken together with the atoms to which they are bonded, form a monocyclic, optionally substituted, aliphatic, heterocyclic, aryl, or heteroaryl ring that is optionally fused to a monocyclic or bicyclic, optionally substituted, aliphatic, heterocyclic, aryl, or heteroaryl ring; R^{e}-R^{j} are independently —H or optionally substituted alkyl; and each of the OSM signaling inhibitor compounds has at least one hydrogen atom bonded to an oxygen, nitrogen, or sulfur atom. The OSM signaling inhibitor compounds also include pharmaceutically acceptable salt or solvates of the compounds represented by Structural Formula (I).

### RELATED APPLICATION

This application claims the benefit of U.S. Provisional Application No. 60/641,041, filed on Jan. 3, 2005. The entire teachings of the above application are incorporated herein by reference.

### BACKGROUND OF THE INVENTION

There are numerous inflammatory disorders needing new methods of treatment. For example, rheumatoid arthritis (RA) is a chronic, destructive, inflammatory autoimmune disease. Structural damage to articular cartilage, in particular the collagen component, represents a critical and irreversible stage of pathogenesis of rheumatoid arthritis. Proinflammatory cytokines, such as interleukin (IL)-1, Oncostatin M (OSM), TNF-α and IL-17 play critical catabolic roles in cartilage destruction. RA causes irreversible joint damage and disability, and reduces life expectancy by an average 3-18 years RA affects approximately 2 million people in the United States (S. E. Gabriel, Rheum Dis Clin North Am, 27, 269 (May, 2001)), with women more prone by a ratio of 3 to 1.

Several disease-modifying antirheumatic drugs (DMARDs) exist, including leflunomide (a pyrimidine synthesis inhibitor), cytokine antagonists, and IL-1 receptor antagonists. Often, combination therapies have emerged.

Despite these advances, however, current treatments are inadequate for a number of reasons. For example, many are no more efficacious in large placebo-controlled trials than methotrexate (MTX). Often, only a small portion of patients achieve improvement, and remissions are exceedingly rare. Moreover, biological agents often require periodic injections which can be expensive, inconvenient, and introduce infection as a risk factor, all of which can lead to patient discontinuation. Further, certain biological agents tend to be unstable after administration.

Therefore, there is still a need for new drugs for treating inflammatory diseases.

### SUMMARY OF THE INVENTION

Disclosed are compositions and methods of treating an inflammatory disorder in a subject, comprising administering an effective amount of an oncostatin M (OSM) and/or IL-1 signaling inhibitor compound. Administration of an effective amount of the OSM signaling inhibitor compounds can inhibit oncostatin M signaling or oncostatin M signal production in a subject in need of such inhibition. The compounds effectively inhibit OSM signaling in cell screens, including screens that employ human cartilage cells as a model for inflammatory diseases such as rheumatoid arthritis (see Examples 1 and 2) or osteoarthritis. The compounds also ameliorate the development of arthritis in a murine collagen-induced arthritis (CIA) model (see Example 12).

The OSM signaling inhibitor compounds are represented by Structural Formula (I):

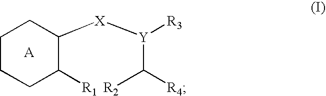

Ring A is optionally substituted, contains zero, one, two, or three double bonds, and is optionally fused to an aliphatic, aryl or heteroaryl ring.

X is an optionally substituted 1 to 3 carbon aliphatic chain that is optionally fused to a monocyclic, optionally substituted, aliphatic, heterocyclic, aryl, or heteroaryl ring, wherein one or two carbons in X are optionally replaced with —O—, —S—, or —NR^{e}—.

Y is carbon or nitrogen.

R₁ and R₂ are independently —H, —OH, —CN, —NO₂, —NR^{f}R^{g}, halogen, optionally substituted alkyl, or optionally substituted alkoxy; or R₁ and R₂ together link the carbons to which they are bonded with a bond, —O—, —S—, or —N^{h}—.

R₃ and R₄ are independently —H, —OH, —CN, —NO₂, —NR^{f}R^{g}, halogen, optionally substituted alkyl, or optionally substituted alkoxy, or R₄ is ═O; or R₃ and R₄, taken together with the atoms to which they are bonded, form a monocyclic, optionally substituted, aliphatic, heterocyclic, aryl, or heteroaryl ring that is optionally fused to a monocyclic or bicyclic, optionally substituted, aliphatic, heterocyclic, aryl, or heteroaryl ring.

R^{e}-R^{j} are independently —H or optionally substituted alkyl.

In certain embodiments the present invention is a method of treating an inflammatory disorder in a subject in need thereof, comprising administering to the subject an effective amount of a compound represented by the following structural formula:

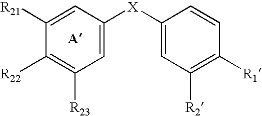

R₁′ and R₂′ are each independently —F, —Cl, —Br, —I, —CN, —NO₂, —OR^{a}, —OC(O)R^{a}, —C(O)OR^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, ═O, ═S, ═CR^{a}R^{b}, ═NR^{a}, ═NOR^{a}, ═NNR^{a}, —OPO₃R^{x}, —NR^{a}SO₂R^{c} or optionally substituted alkyl.

R^{a}-R^{d} are each independently —H or an optionally substituted aliphatic, optionally substituted cycloaliphatic, optionally substituted heterocyclic, optionally substituted benzyl, optionally substituted aryl, or optionally substituted heteroaryl, or, —N(R^{a}R^{b}), taken together, is an optionally substituted heterocyclic group.

Each R^{x} is independently halo, —H, an optionally substituted aliphatic, optionally substituted cycloaliphatic, optionally substituted heterocyclic, optionally substituted benzyl, optionally substituted aryl, or optionally substituted heteroaryl, or, —N(R^{a}R^{b}), taken together, is an optionally substituted heterocyclic group.

R₂₁, R₂₂, and R₂₃ are each independently —H, —OH, —F, —Cl, —Br, or alkoxy; or R₂₁ and R₂₂ together are a methylene dioxy or ethylene dioxy group forming an optionally substituted 5 or 6 member ring fused to the aryl ring to which they are bonded.

In certain embodiments the present invention is a compound represented by the following structural formula:

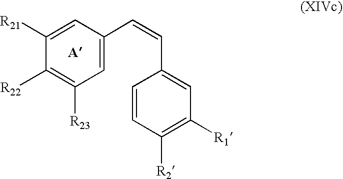

R₁′ and R₂′ are each independently OR^{a}, —OPO₃R^{x}, —NR^{a}SO₂R^{c} or optionally substituted alkyl.

R₂₁, R₂₂, and R₂₃ are independently —H, —OH, —F, —Cl, —Br, or alkoxy; or R₂₁ and R₂₂ together are a methylene dioxy or ethylene dioxy group forming an optionally substituted 5 or 6 member ring fused to the aryl ring to which they are bonded.

Also, in certain embodiments of the present invention each of the OSM or IL-1 signaling inhibitor compounds described herein has at least one hydrogen atom bonded to an oxygen, nitrogen, or sulfur atom. The OSM signaling inhibitor compounds also include pharmaceutically acceptable salts or solvates of the compounds described herein.

In some embodiments, the invention is a method of inhibiting oncostatin M signal production in a subject in need of such inhibition, comprising administering an effective amount of the compound represented by Structural Formula (I).

In some embodiments, the invention is a method of inhibiting MMP-13 expression in a subject in need of such inhibition, comprising administering an effective amount of the compound represented by Structural Formula (I).

The compounds, compositions (for example, pharmaceutical compositions), and methods described herein are believed to be effective for treating the inflammatory diseases described herein, in particular rheumatoid arthritis. In certain embodiments the compounds, compositions (for example, pharmaceutical compositions), and methods described herein are believed to be effective for treating the inflammatory diseases described herein, in particular rheumatoid arthritis.

### BRIEF DESCRIPTION OF THE DRAWINGS

FIG. 1 is a table showing physiological functions of the polyfunctional cytokine OSM that are shared by other IL-6 family members.

FIGS. 2A-E are charts showing that 4A1 responds specifically to OSM stimulation in a time- and dose-dependent manner.

FIG. 2A is a bar graph showing the specific response of the 4A1 cell line to OSM as measured by luciferase reporter activity in comparison to a panel of 17 other ligands.

FIGS. 2B-C are log-log plots showing that luciferase reporter activation (Y-axis) was corroborated by green fluorescent protein (GFP) reporter (X-axis) by fluorescence-activated cell sorting (FACS) analysis.

FIGS. 2D-E are a pair of graphs showing time- and dose-dependent activation of reporter activity by OSM. FIG. 2D shows the increase in reporter activity versus time at an OSM concentration of 10 nanograms/milliliter (ng/mL). FIG. 2E shows the response (in luciferase/10,000 cells) versus OSM concentration in ng/mL.

FIGS. 3A-B demonstrate the inhibitory activity of OSM signaling inhibitors (1)-(11) in the 4A1 cell line. Each IC₅₀ value is the (micromolar, μM) concentration of OSM signaling inhibitor that inhibits OSM-stimulated (luciferase) reporter activity by ≧50%. FIG. 3A is a graph showing the concentration of OSM signaling inhibitors (1) and (11) versus normalized (luciferase) reporter activity. FIG. 3B is a table of IC₅₀ values for OSM signaling inhibitors (1)-(11).

FIGS. 4A-B demonstrate the inhibitory activity of OSM signaling inhibitors (1), (2), (8), (9), and (12) against MMP-13 production in a secondary screen in chondrocyte cell line SW-1353. FIG. 4A is a graph showing the concentration of OSM signaling inhibitor (μM) versus MMP-13 expression (picograms/10,000 cells). FIG. 4B is a table of IC₅₀ values for MMP-13 inhibition by OSM signaling inhibitors (1), (2), (8), (9), and (12).

FIG. 5 is a series of three bar graphs that show OSM signaling inhibitors (1), (2), (8), (9), and (12) preferentially inhibit MMP-13 expression compared to MMP-2 expression.

FIGS. 6A-6D illustrate stilbene derivatives Combretastatin A-1, Piceatannol, trans-Combretastatin A-4 and dihydrocombretastatin A-4 (from Cushman et al., infra).

FIGS. 7A and 7B illustrate orally active heterocycle-based Combretastatin A-4 analogs (from Wang et al., infra).

FIGS. 8A-8C Combretastatin A-4 analogs (from Liou et al., infra).

FIG. 9A is a bar graph showing the specific response of a 4A1 cell line to OSM and IL-1 in comparison to a panel of cytokines/chemicals. Each bar represents the reporter activity as a relative fold induction over unstimulated controls after 31 hours of incubation.

FIGS. 9B and 9C are a pair of graphs showing dose-dependent induced reporter activity of 4A1 by OSM and IL-1β with EC50 of 46±8 ng/ml and 28±10 pg/ml respectively.

FIG. 9D is a bar graph showing modestly increased reporter activity of 4A1 from simultaneous addition of OSM and IL-1 compared with the sum of the separate additions of OSM and IL-1.

FIGS. 10A through 10E are five graphs showing that OSM-stimulated reporter activity is blocked by inhibitors of signaling pathways in 4A1 cells. 4A1 clone was treated with the indicated panel of known inhibitors: FIG. 10A: JAK Inhibitor I (JAK kinase); FIG 10B: SP600125 (JNK); FIG. 10C: SN203580 (p38); FIG. 10D: Kamebakaurin (NF-κB); and FIG. 10E: PD98059 (MEK).

FIGS. 11A and 11B are a pair of graphs showing that CA4 potently inhibits OSM- and IL-1-activated reporter activity in 4A1 cells. FIG. 1I A: Inhibition of OSM signaling pathway (IC50=5.3±0.5 nM). FIG. 11B: Inhibition of IL-1 pathway (IC50=6.9±0.4 nM).

FIGS. 12A through 12E are five graphs showing that CA4 inhibits pro-MMP-13, but not MMP-2 production in SW1353 cells. FIG. 12A: CA4 inhibited OSM/IL-1 induced expression of MMP-13 (IC50=5.6±1.3 nM), the inhibitory effect was also observed with IL-1, IC50=5.2±1.9 nM (FIG. 12B). FIG. 12C: CA4 dose-dependently inhibited OSM-induced reporter activity in a SW1353 Sentinel line and FIG. 12D: OSM-induced pro-MMP-13 production in dedifferentiated primary chondrocytes. FIG. 12E: CA4 failed to inhibit MMP-2 production.

FIG. 13A is a bar graph and FIGS. 13B through 13E are four graphs showing transcriptional repression of MMP-13 mRNA by CA4 and signaling pathways critical to OSM/IL-1-induced pro-MMP-13 production. FIG. 13A: CA4 represses OSM/IL-1-induced MMP-13 mRNA levels by Taqman. FIGS. 13B-E: show that JAK/STAT (JAK Inhibitor I), p38 (SB203580), NF-κB (Kamebakaurin), and JNK (SP600125) pathways were important to OSM/IL-1-stimulated MMP-13 production.

FIGS. 14A through 14D illustrate that CA4 does not affect JAK/STAT, p38, or NF-κB pathways in SW1353 cells. FIG. 14A and FIG. 14B show flow cytometric analysis of phospho Stat-1 (pY701) and p38 (pT180/pY182). FIG. 14C shows that nuclear translocation of Stat1 proteins was investigated using immunohistochemisty. FIG. 14D shows NF-κB reporter activity under indicated treatment conditions.

FIGS. 15A through 15C are three graphs illustrating that CA4 ameliorates development of arthritis symptoms in mouse paws in mouse CIA model. FIG. 15A: CA4 dose-dependently reduced arthritis incidence rate. FIG. 15B: CA4 significantly reduced macroscopic arthritis clinical scores. Asterisks (*) indicate statistical significance (p<0.05). FIG. 15C: Body weight information of CA4 treatment.

FIGS. 16a through 16e illustrate that CA4 protects knee joints in mouse CIA model. Histophathological examination of knee joints of normal mice FIG. 16a; and mice treated with dexamethasone daily at 0.2 mg/kg FIG. 16b; vehicle FIG. 16c; CA4 at 25 mg/kg/day by osmotic pumps FIG. 16d; and CA4 at 16 mg/kg/day by osmotic pumps FIG. 16e. Arrows indicate articular cartilage areas. S: synovial; Mn: meniscus; M: medial; L: lateral; P: panus. Magnification was 100×.

FIGS. 17A through 17C are three bar graphs illustrating that CA4 reduces plasma levels of IL-1β FIG. 17A, Rheumatoid Factors (RF) FIG. 17B, and anti-collagen II antibodies (anti-CII) FIG. 17C, in a mouse CIA model. Plasma from mice without immunization (normal) and mice treated with dexamethasone (Dex), vehicle (Veh), and two doses of CA4 (25, 16 mg/kg/day) were examined by respective ELISAs. Asterisks (*) indicate statistical significance (p<0.05).

FIG. 18 is a schematic representation of the synthesis of compounds represented by (XIVc) of the invention

### DETAILED DESCRIPTION OF THE INVENTION

A description of preferred embodiments of the invention follows.

It has now been discovered that combretastatin A-4 (CA-4, or CA4) is a potent inhibitor of both OSM and IL-1 mediated reporter activity. A reporter cell line (4A1) was established that responded specifically to OSM and IL-1. Using this cell line, CA4 was identified as a potent inhibitor of the reporter activity, and confirmed in cultured chondrocytes that CA4 potently inhibited OSM and IL-1 signal transduction that led to MMP-13 expression. Such inhibition appeared to be selective in that CA4 had no effect on TGF-β- and CD3-mediated Sentinel reporter activities or MMP-2 production in SW1353 cells. In a murine CIA model, CA4 protected articular cartilage and bone resorption, and reduced the levels of arthritis and inflammation biomarkers in blood. It is demonstrated in Example 12 that CA4 protected articular cartilage and bone resorption, and reduced the plasma levels of arthritis and inflammatory markers, in a murine CIA model.

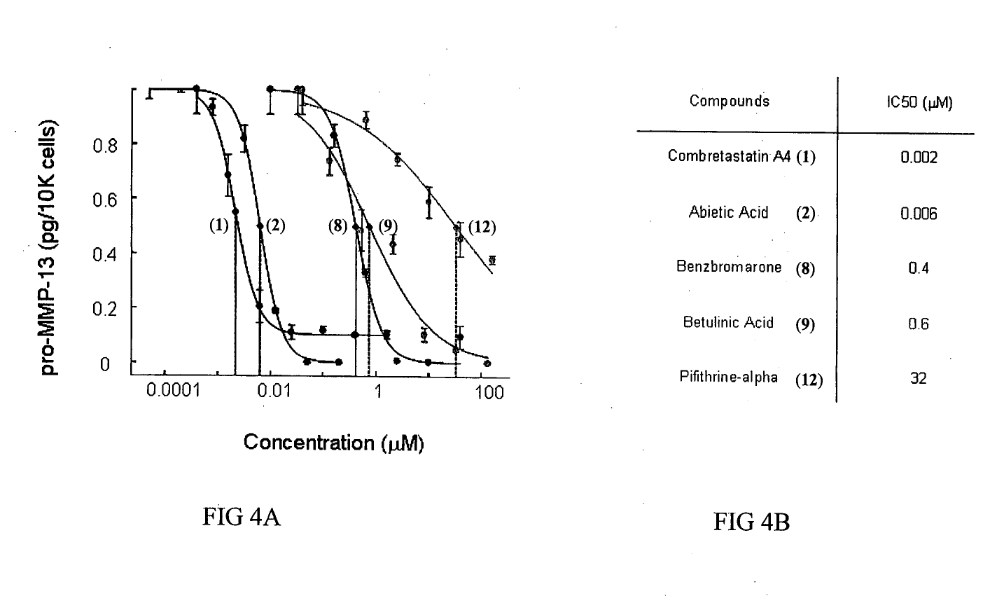

Combretastatin A-4 (CA4, FIG. 6A) is a cis-stilbene first isolated from African tree Combretum caffrum. Without wishing to be bound by theory it is believed that it binds tubulin at or near the colchicine site and disrupts tubulin polymerization, and therefore is among the family of small molecules that are called microtubule interfering agents (MIAs). Tested extensively in many tumor cell lines, CA4 demonstrates potent antiproliferation activity. CA4, or its phosphate prodrug, CA4P, is also an antivascular agent in that it promotes G2/M cell cycle arrest, morphological changes, and eventually cell death of endothelial cells in vivo and results in extensive shutdown of established vasculature in tumors in vivo, leading to constricted tumor blood flow and tumor necrosis. This antivascular property of CA4/CA4P has been employed to reduce retinal neovascularization as well. However, whether CA4 exerts all of these activities solely through microtubules remains to be clarified. While a number of CA4 analogs in structure-activity relationship (SAR) studies demonstrate correlation in terms of potency between its anti-tubulin polymerization and antiproliferation activities, CA4 analogues do exist that have potent antiproliferation activity but weak antitubulin polymerization activity, and vise versa. Lack of correlation between in vivo antiproliferation activity and in vivo antivascular activity has also been reported. These observations leave open the possibility that non-tubulin molecular targets of CA4 might exist.

In certain embodiments the present invention is a method of treating an inflammatory disorder in a subject in need thereof, comprising administering to the subject an effective amount of a compound described herein.

In certain embodiments, the present invention is a method of inhibiting proinflammatory cytokine activity in a subject in need thereof, comprising administering to the subject an effective amount of a compound described herein.

In various embodiments, the compound is represented by Structural Formula (II): 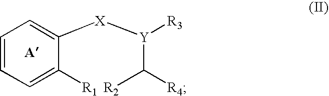
wherein Ring A′ is optionally substituted and is optionally fused to a monocyclic aliphatic, aryl or heteroaryl ring.

In preferred embodiments, the compound is represented by Structural Formula (III): 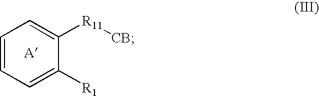
wherein R₁₁ is an optionally substituted C₃-C₁₂ aliphatic chain that is optionally interrupted by —O—, —S—, or —NR^{k}—; wherein R^{k} is —H or optionally substituted alkyl; and CB is a carboxylic acid derivative or a bioisostere thereof. In more preferred embodiments, the compound can be represented by Structural Formula (IV): 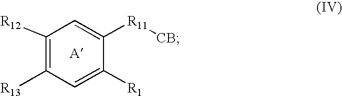
wherein R₁₂ and R¹³ are independently —OR^{a}, —C(O)R^{a}, —OC(O)R^{a}, —C(O)OR^{a}, —N(R^{a}R^{b}), —C(O)N(R^{a}R^{b}), or optionally substituted alkyl, or, together with the carbons of Ring A′ to which they are bonded, form a 5 or 6 membered heterocycle; and R₁₁ is an optionally substituted C4-C8 alkene. The compound can be substituted at one or more substitutable positions with one or more substituents selected from —F, —Cl, —Br, —I, —CN, —NO₂, —OR^{a}, —OC(O)R^{a}, —C(O)OR^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, ═O, ═S, ═CR^{a}R^{b}, ═NR^{a}, NOR^{a}, ═NNR^{a}, C1-C4 alkyl, and C1-C4 haloalkyl. More preferably, the compound can be represented by Structural Formula (V): 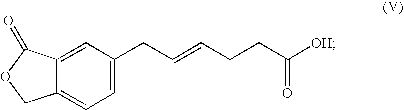
wherein the compound is substituted at one or more substitutable positions with one or more substituents selected from —F, —Cl, —Br, —I, —CN, —NO₂, —OR^{a}, —C(O)OR^{a}, —(N(R^{a}R^{b}), —C(O)N(R^{a}R^{b}), —NR^{c}C(O)R^{a}, —NR^{c}C(O)OR^{a}, —CR^{c}═CR^{a}R^{b}, ═O, ═S, ═CR^{a}R^{b}, ═NR^{a}, NOR^{a}, ═NNR^{a}, C1-C4 alkyl, and C1-C4 haloalkyl. In a preferred embodiment, the compound is 6-(4-Hydroxy-6-methoxy-7-methyl-3-oxo-5-phthalanyl)-4-methyl-4-hexenoic acid (mycophenolic acid, OSM signaling inhibitor (3)):

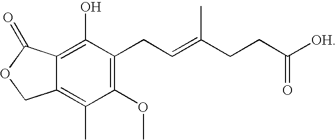

In other preferred embodiments, the compound is represented by Structural Formula (VI): 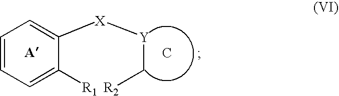
wherein Ring C is an optionally substituted 5 to 12 membered, monocyclic or bicyclic, aliphatic, heterocyclic, aryl, or heteroaryl ring. In other embodiments, the compound can be represented by Structural Formula (VII): 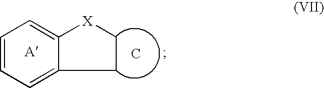
wherein Ring C has 2 or 3 double bonds; one of X and Ring A′ is substituted with a carboxylic acid derivative or a bioisostere thereof; and the compound is substituted at one or more substitutable positions with one or more substituents selected from —F, —Cl, —Br, —I, —CN, —NO₂, —OR^{a}, —OC(O)R^{a}, —C(O)OR^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, ═O, ═S, ═CR^{a}R^{b}, ═NR^{a}, ═NOR^{a}, ═NNR^{a}, and optionally substituted alkyl. More typically, the compound can be substituted at two or more substitutable positions with one or more substituents selected from —F, —Cl, —Br, —I, —CN, —NO₂, —OR^{a}, —C(O)OR^{a}, —N(R^{a}R^{b}), —C(O)N(R^{a}R^{b}), —NR^{c}C(O)R^{a}, —NR^{c}C(O)OR^{a}, —CR^{c}═CR^{a}R^{b}, ═O, ═S, ═CR^{a}R^{b}, ═NR^{a}, ═NOR^{a}, ═NNR^{a}, C1-C4 alkyl, and C1-C4 haloalkyl. In some embodiments, the compound is represented by Structural Formula (VIII): 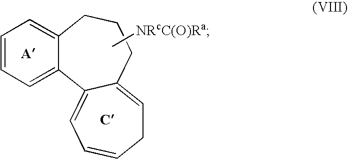
wherein the compound is substituted at two or more substitutable positions with one or more substituents selected from —Cl, —Br, —R^{k}, —OR^{k}, —C(O)OR^{k}, —NHC(O)R^{k}, —NHC(O)OR^{k}, —C(═CH2)R^{k}, ═O, ═CHR^{k}, and ═NR^{k}, wherein R^{k} is methyl, ethyl, propyl, 2-propyl, butyl, sec-butyl, or tertiary butyl. In a preferred embodiment, the compound is colchicine (OSM signaling inhibitor (4)):

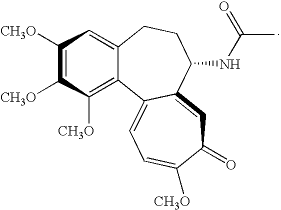

In other preferred embodiments, the compound is represented by Structural Formula (IX): 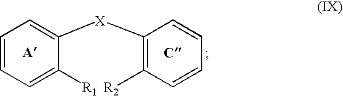
wherein Ring C″ is optionally substituted and is optionally fused to an aliphatic, aryl or heteroaryl ring; at least one of Ring A′ and Ring C″ is substituted with a carboxylic acid derivative or a bioisostere thereof; and the compound is substituted at one or more substitutable positions with one or more substituents selected from —F, —Cl, —Br, —I, —CN, —NO₂, —OR^{a}, —OC(O)R^{a}, —C(O)OR^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, ═O, ═S, ═CR^{a}R^{b}, ═NR^{a}, ═NOR^{a}, ═NNR^{a}, C₁-C₄ alkyl, and C1-C4 haloalkyl. More typically, the compound can be represented by Structural Formula (X): 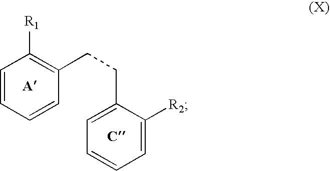
wherein R₁ and R₂ are independently —H, —OH, —CN, —NO₂, —NR^{f}R^{g}, halogen, optionally substituted alkyl, or optionally substituted alkoxy; the compound is substituted at two or more substitutable positions with one or more substituents selected from —F, —Cl, —Br, —I, —CN, —NO₂, —OR^{a}, —C(O)OR^{a}, —N(R^{a}R^{b}), —C(O)N(R^{a}R^{b}), —NR^{c}C(O)R^{a}, —NR^{c}C(O)OR^{a}, —CR^{c}═CR^{a}R^{b}, ═O, ═S, ═CR^{a}R^{b}, ═NR^{a}, ═NOR^{a}, ═NNR^{a}, C₁-C₄ alkyl, and C₁-C₄ haloalkyl; and - - - is a single or double bond. In various embodiments, the compound can be represented by Structural Formula (XI): 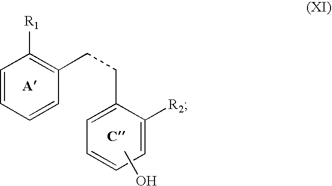
wherein the compound is substituted at one or more substitutable positions with one or more substituents selected from —Cl, —Br, —R^{k}, —OR^{k}, —C(O)OR^{k}, —NHC(O)R^{k}, —NHC(O)OR^{k}, —C(═CH2)R^{k}, ═O, ═CHR^{k}, and ═NR^{k}; wherein R^{k} is methyl, ethyl, propyl, 2-propyl, butyl, sec-butyl, or tertiary butyl. In some preferred embodiments, the compound can be represented by Structural Formula (XII): 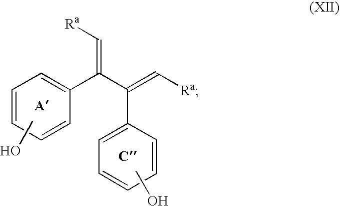
or in a preferred embodiment, the compound is 4-((2E,4E)-4-(4-hydroxyphenyl)hexa-2,4-dien-3-yl)phenol (dienestrol) (OSM signaling inhibitor (10)): 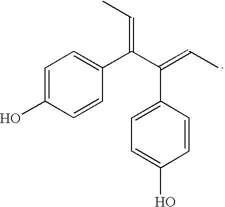
In some preferred embodiments, the compound can be represented by Structural Formula (XIII): 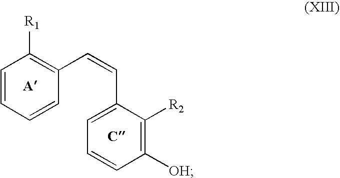
or in a preferred embodiment, the compound is (Z)-5-(3,4,5-trimethoxystyryl)-2-methoxyphenol (combretastatin A4) (OSM signaling inhibitor (1)): 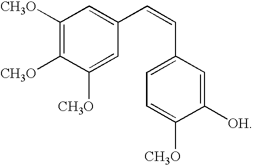
In other preferred embodiments, the compound is represented by Structural Formula (XIV): 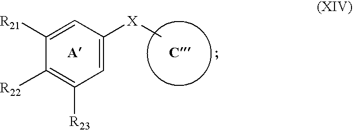
wherein Ring C′″ is an optionally substituted, 5-12 membered, monocyclic or bicyclic, heteroaryl or heterocyclic ring; R₂₁, R₂₂, and R₂₃ are independently —H, —OH, —F, —Cl, —Br, or alkoxy; or R₂₁, and R₂₂ together are a methylene dioxy or ethylene dioxy group forming a 5 or 6 member ring fused to the aryl ring to which they are bonded; and the compound is substituted at one or more substitutable positions with one or more substituents selected from —F, —Cl, —Br, —I, —CN, —NO₂, —OR^{a}, —OC(O)R^{a}, —C(O)OR^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —C(NR^{c})—N(R^{a}R^{b}), —NR^{d} C(NR^{c})—N(R^{a}R^{b}), —NR^{a}N(R^{a}R^{b}), —CR^{c}═CR^{a}R^{b}, ═O, ═S, ═CR^{a}R^{b}, ═NR^{a}, ═NOR^{a}, ═NNR^{a}, C1-C4 alkyl, and C1-C4 haloalkyl.
In some embodiments, the compound can be represented by Structural Formula (XV): 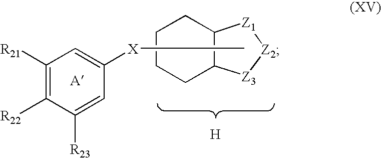
wherein Z₁, Z₂, and Z₃ are each independently C, N, S, or O, provided that at least one of Z₁, Z₂, and Z₃ is N, S, or O, and at least one is C; optionally substituted bicyclic ring H is saturated or unsaturated; and the compound is substituted at one or more substitutable positions with one or more substituents selected from —F, —Cl, —Br, —I, —CN, —NO₂, —OR^{a}, —C(O)OR^{a}, —N(R^{a}R^{b}), —C(O)N(R^{a}R^{b}), —NR^{c}C(O)R^{a}, —NR^{c}C(O)OR^{a}, —CR^{c}═CR^{a}R^{b}, ═O, ═S, ═CR^{a}R^{b}, ═NR^{a}, ═NOR^{a}, ═NNR^{a}, C1-C4 alkyl, and C1-C4 haloalkyl. More typically, Z₁ and Z₃ can be independently N, S, or O. In preferred embodiments, the compound is represented by Structural Formula (XVI): 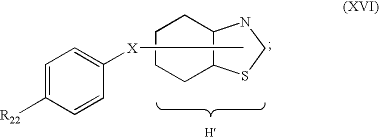
wherein ring H′ is unsaturated and is substituted with at least one substituent selected from —F, —Cl, —Br, —I, —CN, —NO₂, —OR^{a}, —C(O)OR^{a}, —N(R^{a}R^{b}), —C(O)N(R^{a}R^{b}), NR^{c}C(O)R^{a}, —NR^{c}C(O)OR^{a}, —CR^{c}═CR^{a}R^{b}, ═O, ═S, ═CR^{a}R^{b}, ═NR^{a}, ═NOR^{a}, ═NNR^{a}, C1-C4 alkyl, and C1-C4 haloalkyl. More typically, X can be C1-C3 alkyl optionally substituted with ═O; or in a preferred embodiment, the compound is [2-(2-imino-4,5,6,7-tetrahydro benzothiazol-3-yl)-1-p-tolylethanone] (pifithrin alpha) (OSM signaling inhibitor (12)): 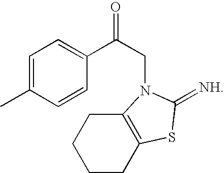
In some embodiments, the compound can be represented by Structural Formula (XVII): 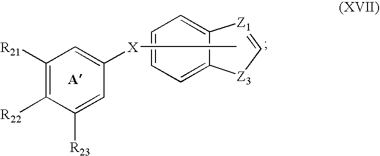
wherein the compound is substituted at one or more substitutable positions with one or more substituents selected from Cl, —Br, —R^{k}, —OR^{k}, —C(O)OR^{k}, —NHC(O)R^{k}, —NHC(O)OR^{k}, —C(═CH2)R^{k}, ═O, ═CHR^{k}, and ═NR^{k}, wherein R^{k} is methyl, ethyl, propyl, 2-propyl, butyl, sec-butyl, and tertiary butyl. More typically, Z₁ and Z₃ can be N. Also, X can typically be —O— or —S—. In a preferred embodiment, the compound is [5-(phenylthio)-1H-benzimidazol-2-yl]carbamic acid methyl ester (Fenbendazole) (OSM signaling inhibitor (6)): 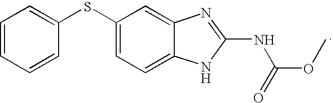
In other preferred embodiments of the compound represented by Structural Formula XVII, Z₁ is N or C, Z₃ is S or O, and R₂₂ is —OH. X can be C1-C3 alkyl optionally substituted with ═O. In a preferred embodiment, the compound is (3,5-dibromo-4-hydroxyphenyl)(2-ethylbenzofuran-3-yl)methanone (benzobromarone) (OSM signaling inhibitor (8)): 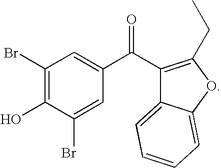
In other preferred embodiments of the compound represented by Structural Formula XIV, Ring C′″ is an optionally substituted, 10 membered, bicyclic heteroaryl group. X can be optionally substituted —CH₂CH₂—, —CH═CH—, —CH₂NH—, or —NHCH₂—. Typically, the compound can be represented by Structural Formula (XVIII): 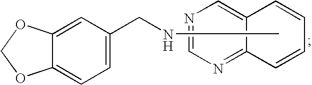
wherein the compound is substituted at one or more substitutable positions with one or more substituents selected from Cl, —Br, —R^{k}, OR^{k}, C(O)OR^{k}, —NHC(O)R^{k}, —NHC(O)OR^{k}, —C(═CH2)R^{k}, ═O, ═CHR^{k}, and ═NR^{k}, wherein R^{k} is methyl, ethyl, propyl, 2-propyl, butyl, sec-butyl, or tertiary butyl. In a preferred embodiment, the compound is 4-[[3,4-(Methylenedioxy)benzyl]amino]-6-chloroquinazoline (MBCQ) (OSM signaling inhibitor (5)): 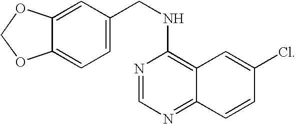
In other preferred embodiments, the compound can be represented by Structural Formula (XIX): 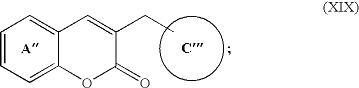
wherein Ring C′″ is an optionally substituted, 5-12 membered, monocyclic or bicyclic, heteroaryl or heterocyclic ring; and the compound is substituted at one or more substitutable positions with one or more substituents selected from —F, —Cl, —Br, —I, —CN, —NO₂, —OR^{a}, —C(O)OR^{a}, —N(R^{a}R^{b}), —C(O)N(R^{a}R^{b}), —NR^{c}C(O)R^{a}, —NR^{c}C(O)OR^{a}, —CR^{c}═CR^{a}R^{b}, ═O, ═S, ═CR R^{b}, ═NR^{a}, ═NOR^{a}, ═NNR^{a}, C1-C4 alkyl, and C1-C4 haloalkyl. In a preferred embodiment, the compound is 4-hydroxy-3-((4-hydroxy-2-oxo-2H-chromen-3-yl)methyl)-2H-chromen-2-one (dicumarol) (OSM signaling inhibitor (11)): 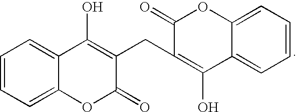
In various preferred embodiments, the compound is represented by Structural Formula (XX): 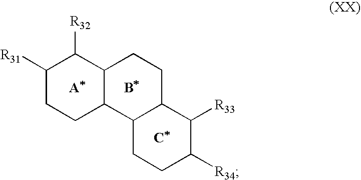
wherein R₃₁ is —H or alkyl and R₃₂ is alkyl; or R₃₁ and R₃₂ together form an optionally substituted 5 or 6 membered aliphatic or heterocyclic ring that is optionally fused to a 5 or 6 membered aliphatic or heterocyclic ring; R₃₃ is —H or alkyl and R₃₄ is alkyl; or R₃₃ and R₃₄ together with the atoms of Ring C* to which they are bonded, form an optionally substituted, 5 or 6 membered, aliphatic or heterocyclic ring that is optionally fused to a 5 to 12 membered, aliphatic or heterocyclic, monocyclic or bicyclic ring; and Rings A*, B*, and C* contain zero, one, or two double bonds, wherein the compound is substituted at one or more substitutable positions with one or more substituents selected from —F, —Cl, —Br, —I, —CN, —NO₂, —OR^{a}, —OC(O)R^{a}, —C(O)OR^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —C(NR^{c})—N(R^{a}R^{b}), —NR^{d}C(NR^{c})—N(R^{a}R^{b}), —NR^{a}N(R^{a}R^{b}), —CR^{c}═CR^{a}R^{b}, ═O, ═S, ═CR^{a}R^{b}, ═NR^{a}, ═NOR^{a}, ═NNR^{a}, C1-C4 alkyl, and C1-C4 haloalkyl. Typically, Ring A* has at least one substituent that is a carboxylic acid derivative or a bioisostere thereof. In some embodiments, the compound is represented by Structural Formula (XXI): 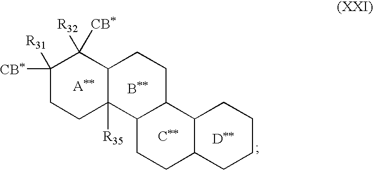
wherein one CB* is —H and one is a carboxylate derivative or bioisostere thereof; R₃₅ is alkyl; optionally substituted Ring D** is optionally fused to a substituted or unsubstituted five or six membered aliphatic ring; and Rings A**, B**, C** and D** each have zero, one, or two double bonds, wherein the compound is substituted at two or more substitutable positions with one or more substituents selected from —F, —Cl, —Br, —I, —CN, —NO₂, —OR^{a}, —C(O)OR^{a}, —N(R^{a}R^{b}), —C(O)N(R^{a}R^{b}), —NR^{c}C(O)R^{a}, —NR^{c}C(O)OR^{a}, —CR^{c}═CR R^{b}, ═O, ═S, ═CR^{a}R^{b}, ═NR^{a}, ═NOR^{a}, ═NNR^{a}, C1-C4 alkyl, and C1-C4 haloalkyl. More preferably, the compound is represented by Structural Formula (XXII): 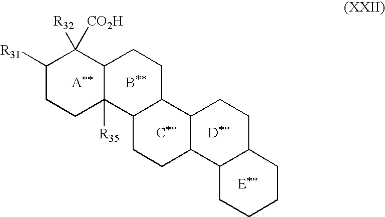
wherein the compound is substituted at one or more substitutable positions with one or more substituents selected from —Cl, —Br, —R^{k}, —OR^{k}, —C(O)OR^{k}, —NHC(O)R^{k}, —NHC(O)OR^{k}, —C(═CH2)R^{k}, ═O, ═CHR^{k}, and ═NR^{k}, wherein R^{k} is methyl, ethyl, propyl, 2-propyl, butyl, sec-butyl, or tertiary butyl; and Ring E** is optionally substituted and has zero, one, or two double bonds. In a preferred embodiment, the compound is 1, 2, 3, 4, 4a, 5, 6, 6a, 6b, 7, 8, 8a, 9, 10, 11, 12, 12a, 14, 14a, 14b-icosahydro-3,4,8a,11,12,14a,14b-heptamethylpicene-4-carboxylic acid (3a-Hydroxy-urs-12-en-23-oic acid, β-boswellic acid) (OSM signaling inhibitor (7)): 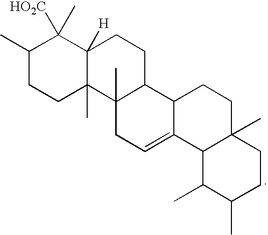
In other preferred embodiments, wherein the compound can be represented by Structural Formula (XXIII): 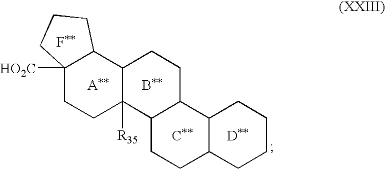
wherein the compound is substituted at one or more substitutable positions with one or more substituents selected from —Cl, —Br, —R^{k}, —OR^{k}, —C(O)OR^{k}, NHC(O)R^{k}, —NHC(O)OR^{k}, —C(═CH2)R^{k}, ═O, ═CHR^{k}, and ═NR^{k}, wherein R^{k} is methyl, ethyl, propyl, 2-propyl, butyl, sec-butyl, or tertiary butyl; and Ring F** is optionally substituted and has zero, one, or 2 double bonds. In a preferred embodiment, the compound is betulinic acid (OSM signaling inhibitor (9)): 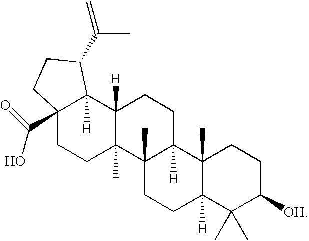
In some preferred embodiments, the compound is represented by Structural Formula (XXIV): 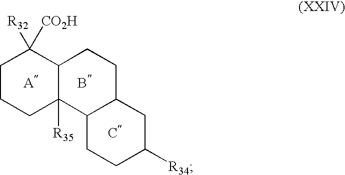
wherein the compound is substituted at one or more substitutable positions with one or more substituents selected from —Cl, —Br, —R^{k}, —OR^{k}, —C(O)OR^{k}, —NHC(O)R^{k}, —NHC(O)OR^{k}, —C(═CH2)R^{k}, ═O, ═CHR^{k}, and ═NR^{k}, wherein R^{k} is methyl, ethyl, propyl, 2-propyl, butyl, sec-butyl, or tertiary butyl. In a preferred embodiment, the compound is (abietic acid) (OSM signaling inhibitor (2)):

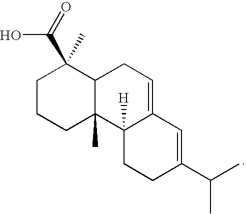

In other preferred embodiments the present invention is a compound represented by Structural Formula (XIVa): 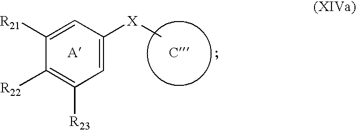
wherein Ring C′″ is an optionally substituted, 5-12 membered, monocyclic or bicyclic, heteroaryl or heterocyclic ring; R₂₁, R₂₂, and R₂₃ are independently —H, —OH, —F, —Cl, —Br, or alkoxy; or R₂₁ and R₂₂ together are a methylene dioxy or ethylene dioxy group forming a 5 or 6 member ring fused to the aryl ring to which they are bonded; and the compound is substituted at one or more substitutable positions with one or more substituents selected from —F, —Cl, —Br, —I, —CN, —NO₂, —OR^{a}, —OC(O)R^{a}, —C(O)OR^{a}, SO₂R^{a}, S₃R^{a}, —PO₂R^{a}R^{b}, —PO₃R^{a}R^{b}, —N(R^{a}R^{b}), —C(O)N(R^{a}R^{b}), C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, NR^{c}C(O)OR^{a}, —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, ═O, ═S, ═CR^{a}R^{b}, ═NR^{a}, ═NOR^{a}, ═NNR^{a}, C1-C4 alkyl, and C1-C4 haloalkyl, or additionally —OPO₃R^{x}, —NR^{a}SO₂R^{c} optionally substituted alkyl.

In certain embodiments the present invention is a compound represented by (XIVb) 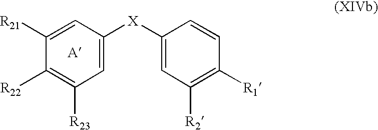
wherein R₁′ and R₂′ are each independently —F, —Cl, —Br, —I, —CN, —NO₂, —OR^{a}, —OC(O)R^{a}, —C(O)OR^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —C(N^{c})—N(R^{a}R^{b}), —NR^{d}—C(NR^{c})—N(R^{a}R^{b}), —NR^{a}N(R^{a}R^{b}), —CR^{c}═CR^{a}R^{b}, ═O, ═S, ═CR^{a}R^{b}, ═NR^{a}, ═NOR^{a}, ═NNR^{a}, C₁-C₄ alkyl, and C₁-C₄ haloalkyl, additional values for R₁′ and R₂′ are independently —OPO₃R^{x}, NR^{a}SO₂R^{c} and optionally substituted alkyl wherein R^{a}-R^{d} are each independently —H or an optionally substituted aliphatic, optionally substituted cycloaliphatic, optionally substituted heterocyclic, optionally substituted benzyl, optionally substituted aryl, or optionally substituted heteroaryl, or, —N(R^{a}R^{b}), taken together, is an optionally substituted heterocyclic group and wherein R^{x} is as defined for R^{a}-R^{d} above and additionally halo.

In certain embodiments the present invention is a compound represented by (XIVc) 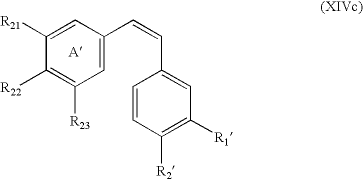
wherein R₁′ and R₂′ are as described above or in certain embodiments R₁′ and R₂′ are each independently —OR^{a}, —OPO₃R^{x}, —NR^{a}SO₂R^{c} or optionally substituted alkyl.

In certain embodiments for XIVc R₂₁-R₂₃ are —OCH₃ and R₁′ is —OH or —OPO₃Na when R₂′ is OCF₃, —OCHF₂, —OCH₂CH₂OCH₃ or —OCH₂(C3 cycloalkyl). In certain other embodiments for XIVc, R₂₁-R₂₃ are —OCH₃ and R₁′ is —CH₂OH or —NHSO₂CH₃ when R₂′ is OCH₃.

In one embodiment, the compound represented by Structural Formula I is not combretastatin A4.

In one embodiment, the compound represented by Structural Formula I is not combretastatin.

In one embodiment, the compound represented by Structural Formula I is not combretastatin A1.

In one preferred embodiment, the compound represented by Structural Formula I is not fenbendazole.

In one preferred embodiment, the compound represented by Structural Formula I is not abietic acid.

In one preferred embodiment, the compound represented by Structural Formula I is not β-boswellic acid.

In one preferred embodiment, the compound represented by Structural Formula I is not mycophenolic acid.

In one preferred embodiment, the compound represented by Structural Formula I is not benzobromarone.

In one preferred embodiment, the compound represented by Structural Formula I is not colchicine.

In one preferred embodiment, the compound represented by Structural Formula I is not betulinic acid.

In one preferred embodiment, the compound represented by Structural Formula I is not 4-[[3,4-(Methylenedioxy)benzyl]amino]-6-chloroquinazoline (MBCQ).

In one preferred embodiment, the compound represented by Structural Formula I is not dienestrol.

In one preferred embodiment, the compound represented by Structural Formula I is not dicumarol.

In one preferred embodiment, the compound represented by Structural Formula I is not pifithrine-α.

In one embodiment the compound or the isostere or the bioisostere is not 1-(4-Methoxy-3-(5-nitrothien-2-yl)methoxy)phenyl-2-(3,4,5-trimethoxy)phenyl-Z-ethene, 1-(4-Methoxy-3-(1-(5-nitrothien-2-yl)ethoxy))phenyl-2-(3,4,5-trimethoxy)phenyl-Z-ethene, 1-(4-Methoxy-3-(5-nitrothien-2-yl)methoxycarbonyloxy)phenyl-2-(3,4,5-trimethoxy)phenyl-Z-ethene, 5-Methoxy-3-((3,4,4′,5-tetramethoxy-(Z)-stilbene-3′-yl)oxy)methyl-1,2-dimethylindole-4,7-dione or 3-((3,4,4′,5-Tetramethoxy-(Z)-stilbene-3′-yl)oxy)methyl-1,2-dimethyl-5-(4-methylpiperazin-1-yl)indole-4,7-dione.

In another embodiment the compound or the isostere or the bioisostere is not doxorubicin, daunorubicin, trimetrexate, methotrexate; etoposide, teniposide, topotecan, SN38, podophyllotoxin, vinblastine, vincristine, vinorelbine, paclitaxel, docetaxel, epirubicin, gefitinib or erlotinib. Additionally the compound or the isostere or the bioisostere is not ZD6474, or epothilone or D AZD2171.

In another embodiment the compound or the isostere or the bioisostere is not 1-(4-Methoxy-3-(2-(5-nitrothiophen-2-yl)propan-2-yl)oxyphenyl-2-(3,4,5-trimethoxy)phenyl-Z-ethene, 1-(4-Methoxy-3-(2-(4-nitrophenyl)propan-2-yl)oxyphenyl-2-(3,4,5-trimethoxy)phenyl-Z-ethene, 6-(2-(4-nitrophenyl)propan-2-ylsulfanyl)-9H-purine, 1-(4-Methoxy-3-(1-methyl-4-(5-nitrothien-2-yl)piperidin-4-yl)oxycarbonyloxy)phenyl-2-(3,4,5-trimethoxy)phenyl-Z ethene, 1-(4-Methoxy-3-(2-(1-methyl-2-nitroimidazol-5-yl)propan-2-yl)oxyphenyl-2-(3,4,5-trimethoxy)phenyl-Z-ethene, 6-(2-(5-nitrothien-2-yl)propan-2-ylsulfanyl)-9H-purine, 1-(3-(1-Ethoxycarbonyl-1-(5-nitrothien-2-yl)ethoxy)-4-methoxy-phenyl)-2-(3,4,5-trimethoxyphenyl)-Z-ethene or N-(2-{3-[1-Methyl-4-(5-nitro-thiophen-2-yl)-ethoxy]-phenyl}-ethyl)-acetamide. Additionally the compound or the isostere or the bioisostere is not 9-(7,8-Dihydroxy-2-methyl-hexahydro-pyrano[3,2-d][1,3]-dioxin-6-yloxy)-5-{3,5-dimethoxy-4-[1-methyl-4-(4-nitrophenyl)-ethoxy]-phenyl}-5,8,8a,9-tetrahydro-5aH-furo[3′,4′:6,7]naphtho[2,3-d][1,3]dioxol-6-one.

In one preferred embodiment, the compound represented by Structural Formula I is not combretastatin A4, fenbendazole, abietic acid, β-boswellic acid, mycophenolic acid, benzobromarone, colchicines, betulinic acid, 4-[[3,4-(Methylenedioxy)benzyl]amino]-6-chloroquinazoline (MBCQ), dienestrol, dicumarol, or pifithrine-α.

In one preferred embodiment, the compound represented by Structural Formula I is selected from combretastatin A4, fenbendazole, abietic acid, β-boswellic acid, mycophenolic acid, benzobromarone, colchicines, betulinic acid, 4-[[3,4-(Methylenedioxy)benzyl]amino]-6-chloroquinazoline (MBCQ), dienestrol, dicumarol, and pifithrine-α.

In one preferred embodiment, the compound represented by Structural Formula I is selected from fenbendazole, abietic acid, β-boswellic acid, mycophenolic acid, benzobromarone, colchicines, betulinic acid, 4-[[3,4-(Methylenedioxy)benzyl]amino]-6-chloroquinazoline (MBCQ), dienestrol, dicumarol, and pifithrine-α.

The compounds represented by Structural Formula I also include various analogs of combretastatin A4, for example, as described in Cushman, M et al., J. Med. Chem., 1991, 34, 2579-2588; Wang, L et al., J. Med. Chem., 2002, 45, 1697-1711; Liou, J-P et al., J. Med. Chem., 2004, 47, 4247-4257. The entire teachings of each of these documents is incorporated herein by reference.

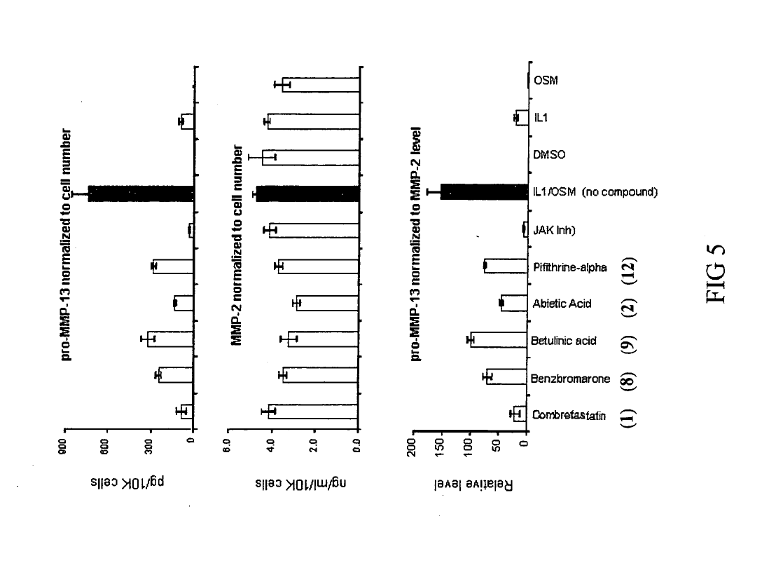

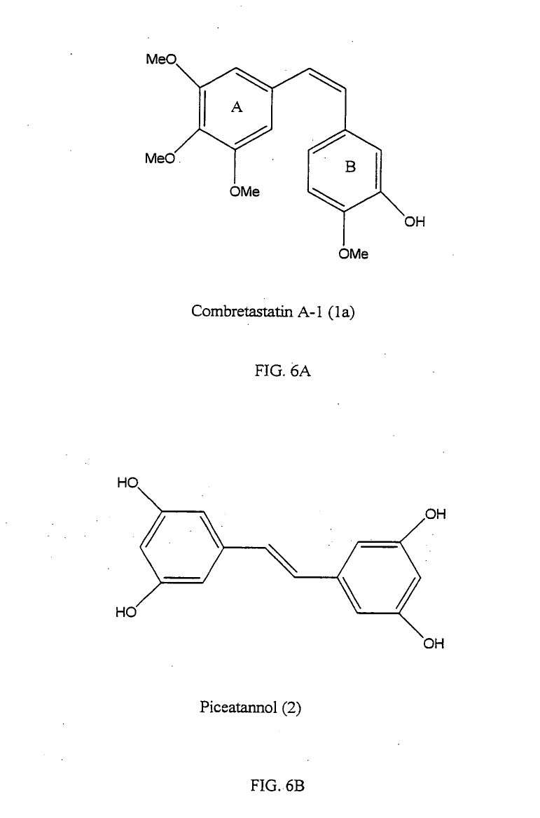

In some preferred embodiments of the invention, the administered compounds are analogs, derivatives or formulations of the OSM signaling inhibitors (e.g., CA-4) disclosed herein. In a preferred embodiment, the compounds are those disclosed in Cushman et al., supra, Wang et al., supra, and Liou et al., supra. In one preferred embodiment, the compounds are those provided in FIGS. 6A-6D herein, and other compounds disclosed in Cushman et al. In one preferred embodiment, the compounds are those provided in FIGS. 7A-7B herein, and other compounds disclosed in Wang et al. In one preferred embodiment, the compounds are those provided in FIGS. 8A-8C herein, and other compounds disclosed in Liou et al. Specifically encompassed as a preferred embodiment of this invention is treatment of any inflammatory disorder described herein using any such compound disclosed in these references.

In some preferred embodiments of the invention, the administered compounds are derivatives and formulations of any compound disclosed herein. In one preferred embodiment, the compound is not a pro-drug, for example, a pro-drug of CA-4. As defined herein, a “pro-drug” includes a compound which is a modification of a compound (e.g., CA-4) wherein a group is added or modified to alter delivery and/or adenine absorption, distribution, metabolism and/or excretion properties.

An aliphatic group is a straight chained, branched or cyclic non-aromatic hydrocarbon which is completely saturated or which contains one or more units of unsaturation. An alkyl group is a saturated aliphatic group. Typically, a straight chained or branched aliphatic group has from 1 to about 10 carbon atoms, preferably from 1 to about 4, and a cyclic aliphatic group has from 3 to about 10 carbon atoms, preferably from 3 to about 8. An aliphatic group is preferably a straight chained or branched alkyl group, e.g., methyl, ethyl, n-propyl, iso-propyl, n-butyl, sec-butyl, tert-butyl, pentyl, hexyl, pentyl or octyl, or a cycloalkyl group with 3 to about 8 carbon atoms. C₁-C₄ straight chained or branched alkyl or alkoxy groups or a C₃-C₈ cyclic alkyl or alkoxy group (preferably C₁-C₄ straight chained or branched alkyl or alkoxy group) are also referred to as a “lower alkyl” or “lower alkoxy” groups; such groups substituted with —F, —Cl, —Br, or —I are “lower haloalkyl” or “lower haloalkoxy” groups; a “lower hydroxyalkyl” is a lower alkyl substituted with —OH; and the like.

An “alkylene group” is represented by —(CH₂)ₙ—, wherein n is an integer from 1-10, preferably 1-4.

The term “aryl” refers to C6-C14 carbocyclic aromatic groups such as phenyl, biphenyl, and the like. Aryl groups also include fused polycyclic aromatic ring systems in which a carbocyclic aromatic ring is fused to other aryl, cycloalkyl, or cycloaliphatic rings, such as naphthyl, pyrenyl, anthracyl, and the like.

The term “heteroaryl” refers to 5-14 membered heteroaryl groups having 1 or more O, S, or N heteroatoms. Examples of heteroaryl groups include imidazolyl, isoimidazolyl, thienyl, furanyl, fluorenyl, pyridyl, pyrimidyl, pyranyl, pyrazolyl, pyrrolyl, pyrazinyl, thiazoyl, isothiazolyl, oxazolyl, isooxazolyl, 1,2,3-trizaolyl, 1,2,4-triazolyl, imidazolyl, thienyl, pyrimidinyl, quinazolinyl, indolyl, tetrazolyl, and the like. Heteroaryl groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazolyl, benzoisothiazolyl, benzooxazolyl, benzoisooxazolyl, benzimidazolyl, quinolinyl, isoquinolinyl and isoindolyl.

Heterocyclic groups are non-aromatic carbocyclic rings which include one or more heteroatoms such as N, O, or S in the ring. The ring can be five, six, seven or eight-membered. Examples include oxazolinyl, thiazolinyl, oxazolidinyl, thiazolidinyl, tetrahydrofuranyl, tetrahydrothiophenyl, morpholino, thiomorpholino, pyrrolidinyl, piperazinyl, piperidinyl, and thiazolidinyl.

Suitable optional substituents for a substitutable atom in alkyl, cycloalkyl, aliphatic, cycloaliphatic, heterocyclic, benzylic, aryl, or heteroaryl groups are those substituents that do not substantially interfere with the anti-inflammatory pharmaceutical activity of the OSM signaling inhibitors. A “substitutable atom” is an atom that has one or more valences or charges available to form one or more corresponding covalent or ionic bonds with a substituent. For example, a carbon atom with one valence available (e.g., —C(—H)═) can form a single bond to an alkyl group (e.g., —C(-alkyl)=), a carbon atom with two valences available (e.g., —C(H₂)—) can form one or two single bonds to one or two substituents (e.g., —C(alkyl)(Br))—, —C(alkyl)(H)—) or a double bond to one substituent (e.g., —C(═O)—), and the like. Substitutions contemplated herein include only those substitutions that form stable compounds.

For example, suitable optional substituents for substitutable carbon atoms include —F, —Cl, —Br, —I, —CN, —NO₂, —OR^{a}, —C(O)R^{a}, —OC(O)R^{a}, —C(O)OR^{a}, SR^{a}, —C(S)R^{a}, —OC(S)R^{a}, —C(S)OR^{a}, —C(O)SR^{a}, —C(S)SR^{a}, —S(O)R, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b} SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, —C≡CR^{a}, ═O, ═S, ═CR^{a}R^{b}, ═NR^{a}, ═NOR^{a}, ═NNR^{a}, optionally substituted alkyl, optionally substituted cycloalkyl, optionally substituted aliphatic, optionally substituted cycloaliphatic, optionally substituted heterocyclic, optionally substituted benzyl, optionally substituted aryl, and optionally substituted heteroaryl, additional values for substituents on carbon atoms include —OPO₃R^{x} and —NR^{a}SO₂R^{c}; wherein R^{a}-R^{d} are each independently —H or an optionally substituted aliphatic, optionally substituted cycloaliphatic, optionally substituted heterocyclic, optionally substituted benzyl, optionally substituted aryl, or optionally substituted heteroaryl, or, —N(R^{a}R^{b}), taken together, is an optionally substituted heterocyclic group and wherein R^{x} is as defined for R^{a}-R^{d} above and additionally halo.

Suitable substituents for nitrogen atoms having two covalent bonds to other atoms include, for example, optionally substituted alkyl, optionally substituted cycloalkyl, optionally substituted aliphatic, optionally substituted cycloaliphatic, optionally substituted heterocyclic, optionally substituted benzyl, optionally substituted aryl, optionally substituted heteroaryl, —CN, —NO₂, —OR^{a}, —C(O)R^{a}, —OC(O)R^{a}, —C(O)OR^{a}, —SR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), and the like.

A nitrogen-containing heteroaryl or non-aromatic heterocycle can be substituted with oxygen to form an N-oxide, e.g., as in a pyridyl N-oxide, piperidyl N-oxide, and the like. For example, in various embodiments, a ring nitrogen atom in a nitrogen-containing heterocyclic or heteroaryl group can be substituted to form an N-oxide.

Suitable substituents for nitrogen atoms having three covalent bonds to other atoms include —OH, alkyl, and alkoxy (preferably C₁-C₄ alkyl and alkoxy). Substituted ring nitrogen atoms that have three covalent bonds to other ring atoms are positively charged, which is balanced by counteranions such as chloride, bromide, fluoride, iodide, formate, acetate and the like. Examples of other suitable counteranions are provided in the section below directed to suitable pharmacologically acceptable salts.

Typically, the compound represented by Structural Formula I has at least one substituent that is a carboxylic acid derivative or a bioisostere thereof. As used herein, “isosteres” refer to elements, functional groups, substituents, molecules or ions having different molecular formulae but exhibiting similar or identical physical properties. Typically, two isosteric molecules have one or more similarities in their volume, shape, charge or charge distribution, polarizability, ionizability, and the like. Typically, isosteric compounds can be isomorphic and can co-crystallize. Other physical properties that can be similar among isosteric compounds include boiling point, density, viscosity and thermal conductivity. However, not all properties need be identical; certain properties can be different such as dipolar moment, polarity, polarization, volume, shape, and the like. The term “isosteres” encompasses “bioisosteres” which are isosteres that, in addition to their physical similarities, share one or more common biological properties. For example, tetrazole is a bioisostere of carboxylic acid because it can mimic some properties of a carboxylic acid group even though it has a different molecular formula. Typically, bioisosteres interact with the same recognition site or can produce broadly similar biological effects. See, for example, Wermuth, CG “Molecular Variations Based on Isosteric Replacements” pp 203-238, in The Practice of Medicinal Chemistry, Wermuth, C G ed, Academic Press, New York, 2^{nd} Ed, 1996; the entire teachings of which are incorporated herein by reference.

Thus “carboxylic acid bioisosteres” include, for example, direct derivatives such as hydroxamic acids, acyl-cyanamides, and acylsulfonamides; planar acidic heterocycles such as tetrazoles, mercaptoazoles, sulfinylazoles, sulfonylazoles, isoxazoles, isothiazoles, hydroxythiadiazoles, and hydroxychromes (e.g., tetrazole, 1,2,3-triazole, 1,2,4-triazole and imidazole); sulfur- or phosphorus-derived acidic functions such as phosphinates, phosphonates, phosphonamides, sulphonates, sulphonamides, acylsulphonamides, alkylsulfonylcarbamoyl, arylsulfonylcarbamoyl and heteroarylsulfonylcarbamoyl; and the like.

In various embodiments, a group that is a carboxylic acid derivative or bioisostere, thereof can be —OH, —CN, —NO₂, —C(O)R^{k}, —OC(O)R^{a}, —C(O)OR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), ═NR^{a}, ═NOR^{a}, ═NNR^{a}, or optionally substituted tetrazole, mercaptoazole, sulfinylazole, sulfonylazole, isoxazole, isothiazole, hydroxythiadiazole, or hydroxychrome. Generally, a group that is a carboxylic acid derivative or bioisostere thereof can be —OH, —OC(O)R^{a}, —C(O)OR^{a}, —C(S)OR^{a}, —C(O)SR^{a}, —C(S)SR^{a}, —S(O)R^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), ═NR^{a}, or optionally substituted tetrazole, 1,2,3-triazole, 1,2,4-triazole or imidazole. Typically, a group that is a carboxylic acid derivative or bioisostere thereof can be —OH, —C(O)OH, —C(S)OH, —C(O)SH, —C(S)SH, —SO₂H, —SO₃H, —PO₂H₂, —PO₃H₂, —NHR^{a}, —NH—, —C(O)NHR^{a}, —C(O)NHNHSO₂R^{c}, —C(O)NHSO₂R^{c}, —SO₂NHR^{a}, —SO₂NHR^{a}, —NHC(O)R^{a}, —NHC(O)OR^{a}, —NHC(O)NHR^{a}, ═NH, or optionally substituted tetrazole, 1,2,3-triazole, 1,2,4-triazole or imidazole. More typically, a group that is a carboxylic acid derivative or bioisostere thereof is —OH, —CO₂H, —NHC(O)CH₃, —NHC(O)OCH₃, —NHC(O)OCH₃, —NH—, ═NH, tetrazole, 1,2,3-triazole, 1,2,4-triazole or imidazole. Preferably, a group that is a carboxylic acid derivative or bioisostere thereof is —OH (bonded to aryl), —CO₂H, —NHC(O)CH₃, —NHC(O)OCH₃, —NHC(O)OCH₃, or an amine.

In certain embodiments the compounds of the present invention or the OSM signaling inhibitors described herein are also IL-1 signal inhibitors.

The OSM or IL-1 signaling inhibitors described herein and the compounds of the present invention can also be combined with a pharmaceutically acceptable carrier or diluent as part of a pharmaceutical composition for therapy.

The OSM or IL-1 signaling inhibitors described herein and the compounds of the present invention and methods of the present invention can be used to treat subjects (e.g., humans) with inflammatory disorders. As used herein, “inflammatory disorders” include local inflammatory responses and systemic inflammation. Examples of inflammatory disorders include: transplant rejection; chronic inflammatory disorders of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung disorders such as asthma, adult respiratory distress syndrome, and chronic obstructive airway disease; inflammatory disorders of the eye including corneal dystrophy, trachoma, onchocerciasis, uveitis, sympathetic ophthalmitis and endophthalmitis; chronic inflammatory disorders of the gums, including gingivitis and periodontitis; tuberculosis; leprosy; inflammatory diseases of the kidney including uremic complications, glomerulonephritis and nephrosis; inflammatory disorders of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis; autoimmune diseases, immune-complex vasculitis, systemic lupus and erythematodes; systemic lupus erythematosus (SLE); and inflammatory diseases of the heart (such as cardiomyopathy, ischemic heart disease hypercholesterolemia, atherosclerosis); as well as various other diseases with significant inflammatory components, including preeclampsia; chronic liver failure, brain and spinal cord trauma, cancer and AIDS. There may also be a systemic inflammation of the body, exemplified by Gram-positive or Gram-negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent used in cancer chemotherapy.

In various embodiments, the inflammatory disorder includes chronic inflammatory pathologies and vascular inflammatory pathologies, including chronic inflammatory pathologies such as sarcoidosis, chronic inflammatory bowel disease, ulcerative colitis, and Crohn's pathology and vascular inflammatory pathologies, such as disseminated intravascular coagulation, atherosclerosis, and Kawasaki's pathology, rheumatoid arthritis, juvenile rheumatoid arthritis, systemic onset juvenile rheumatoid arthritis, psoriatic arthritis, and the like.

In preferred embodiments, the inflammatory disorder is rheumatoid arthritis (RA). In rheumatoid arthritis, typical symptoms, one or more of which may be treated in various embodiments, include pain, stiffness, early morning stiffness, swelling, tender and swollen joints and loss of function; see, for example, Bennett J C. “The etiology of rheumatoid arthritis”, Textbook of Rheumatology (Kelley W N, Harris E D, Ruddy S, Sledge C B, eds.) W B Saunders, Philadelphia pp 879-886, 1985. RA can lead to cartilage damage, bone-resorption and panus formation.

In certain embodiments, the compounds described herein for use in the methods of the present invention inhibit proinflammatory cytokine activity. In certain embodiments the compound described herein for use in the methods of the present invention is CA4. In certain embodiments the compounds described herein for use in the methods of the present invention, such as, CA4, bind tubulin at or near the colchicines site and disrupt tubulin polymerization. These compounds are referred to herein as microtubule interfering agents (MIAs). In certain these MIAs demonstrate potent antiproliferation activity. In certain other embodiments the compounds described herein for use in the methods of the present invention such as, CA4 or its phosphate prodrug CA4P are antivascular agents. In certain embodiments these compounds promote G2/M cell cycle arrest, morphological changes and eventually cell death of endothelial cells in vitro and result in extensive shutdown of established vasculature in tumors in vivo, leading to constricted tumor blood flow and tumor necrosis. The antivascular property of the compounds described herein for use in the methods of the present invention, such as, CA4/CA4P can be employed to reduce retinal neovascularization as well. In certain embodiments the compounds described herein for use in the methods of the present invention target other than tubulin molecular targets.

Oncostatin M in Rheumatoid Arthritis

Without wishing to be bound by theory, it is helpful to review the current understanding of mechanisms believed to be important in inflammatory disorders such as rheumatoid arthritis.

For example, the proinflammatory cytokine oncostatin M (OSM) is a 28-kD glycoprotein of the gp130-binding cytokine family (IL-6 family), which also includes IL-6, cardiotrophin 1 (CT-1), IL-11, Leukemia inhibitory factor (LIF), ciliary neurotrophic factor (CNTF), and cardiotrophin-like cytokine (CTC). OSM is produced mainly by activated T-lymphocytes, monocytes, macrophages, and endothelial cells (J. P. Pelletier, J. Martel-Pelletier, Arthritis Rheum 48, 3301 (December, 2003); S. L. Grant, C. G. Begley, “The Oncostatin M Signaling Pathway: Reversing the Neoplastic Phenotype,” Molecular Medicine Today 5(9): 406-412 (1999). In RA synovium, OSM is primarily produced by macrophages (H. Okamoto et al., Arthritis Rheum 40, 1096 (June, 1997)).

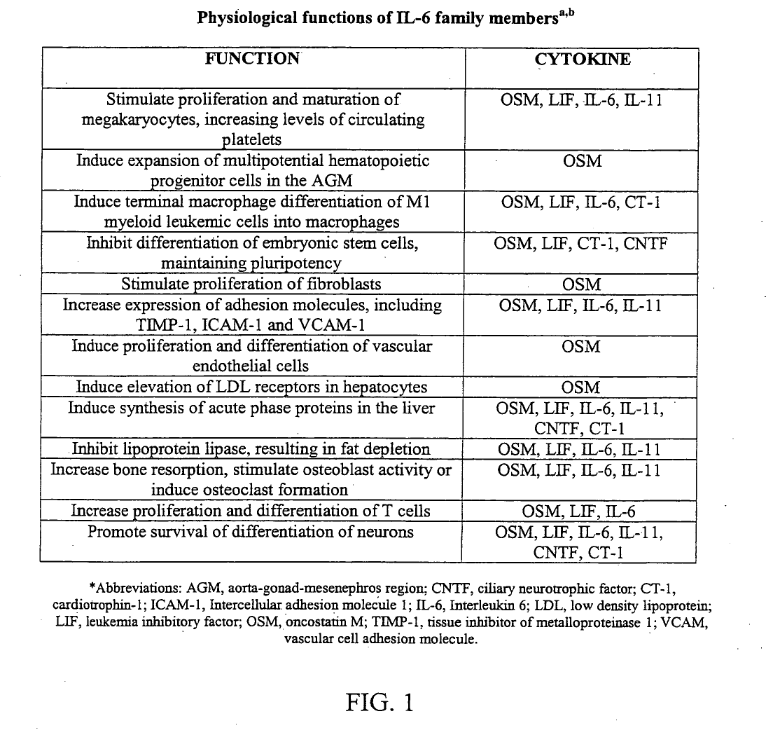

FIG. 1 is a table showing physiological functions of the polyfunctional cytokine OSM that are shared by other IL-6 family members. These functions can include events during inflammation, and effects on hematopoietic tissues, bone, hepatocytes, neurons, and malignancy.

OSM signaling can be mediated by the gp130 receptor family and JAK/STAT and MAP kinase pathways (Grant, 1999). Specifically, human OSM is believed to signal through heterodimers of gp130 and LIFR_{β} (type I, also used by human LIF), and heterodimers of gp130 and OSMR_{β} (type II, OSM-specific). Murine OSM is believed to signal only through the type II receptor complex.

Evidence that is believed to implicate OSM in RA comes from human patients, animal models, and ex vivo and in vitro studies. OSM can be detected in the synovial fluid (SF) of RA patients at elevated levels compared to those in osteoarthritis patients (Okamoto, 1997; D. H. Manicourt et al., Arthritis Rheum 43, 281 (February, 2000); W. Hui, M. Bell, G. Carroll, Ann Rheum Dis 56, 184 (March, 1997). Moreover, the levels of OSM can correlate strongly with those of antigenic keratin sulfate and pyridinoline, believed to be markers of breakdown products of connective tissues in SF from RA patients (Manicourt, 2000).

Multiple reports indicate in vivo involvement of OSM in animal models of arthritis. Elevated OSM mRNA levels can be observed in a murine collagen-induced arthritis (CIA) model (C. Plater-Zyberk et al., Arthritis Rheum 44, 2697 (November, 2001)). Moreover, introarticular injection of human OSM and mouse OSM into goat and mouse joints, respectively, or injection of an adenoviral construct expressing murine OSM into a mouse joint can induce inflammation and cartilage destruction (C. Langdon et al., Am J Pathol 157, 1187 (October, 2000); M. C. Bell, G. J. Carroll, H. M. Chapman, J. N. Mills, W. Hui, Arthritis Rheum 42, 2543 (December, 1999); A. S. de Hooge et al., Arthritis Rheum 48, 1750 (June, 2003)). Moreover, antibodies against OSM can profoundly inhibit joint inflammation and cartilage damage in murine CIA and PIA (pristane-induced arthritis) models, which can result in substantially reduced clinical disease severity (Plater-Zyberk, 2001). It is also believed OSM can contribute both early and crucially to the pro-inflammatory cascade in CIA (Plater-Zyberk, 2001).

OSM is believed to synergize with IL-1, and IL-17, resulting in cartilage destruction, and proteoglycan and collagen degradation (A. D. Rowan et al., Arthritis Rheum 44, 1620 (July, 2001); P. J. Koshy et al., Ann Rheum Dis 61, 704 (August 2002); P. J. Koshy et al., Arthritis Rheum 46, 961 (April, 2002); and W. Hui, A. D. Rowan, C. D. Richards, T. E. Cawston, Arthritis Rheum 48, 3404 (December, 2003)). This synergy is believed to be restricted to OSM, and not observed with any other cytokine members of the family. The synergistic effects are believed to be through the induction of expression of collagenases, stromelysin 1, and one of the aggrecanases, ADCAM-TS4 (Koshy, 2002). In addition, OSM is also believed to induce proinflammatory mediators, such as prostaglandin E2 (PGE2), in several cell types (P. Repovic, K. Mi, E. N. Benveniste, Glia 42, 433 (June, 2003); T. Lahiri, J. D. Laporte, P. E. Moore, R. A. Panettieri, Jr., S. A. Shore, Am J Physiol Lung Cell Mol Physiol 280, L1225 (June, 2001); D. A. Knight et al., Br J Pharmacol 131, 465 (October, 2000); C. D. Richards, A. Agro, Cytokine 6, 40 (January, 1994)). Thus, the OSM signaling pathway may be important to the cartilage destruction component of RA pathogenesis.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The terms “treat” and “treatment,” as used herein, refer to the alleviation, e.g., amelioration of one or more symptoms or effects associated with the disease, prevention, inhibition or delay of the onset of one or more symptoms or effects of the disease, and/or lessening of the severity or frequency of one or more symptoms or effects of the disease, such as the symptoms and effects described herein.

The terms “improve”, “increase” or “reduce,” as used herein, indicate values that are relative to a baseline measurement, such as a measurement in the same individual prior to initiation of the treatment described herein, or a measurement in a control individual (or multiple control individuals) in the absence of the treatment described herein. A control individual is an individual afflicted with the same disorder as the individual being treated, who is about the same age as the individual being treated (to ensure that the stages of the disease in the treated individual and the control individual are comparable).

An “effective amount” is the quantity of compound in which a beneficial clinical outcome is achieved when the compound is administered to a subject in need of treatment. The compound or additional therapeutic agent can be administered in an “effective amount” (i.e., a dosage amount that, when administered at regular intervals, is sufficient to treat the disease, such as by ameliorating symptoms associated with the disease, preventing or delaying the onset of the disease, and/or also lessening the severity or frequency of symptoms of the particular inflammatory disorder, as described above). Thus, an effective amount of the agents or compositions of the invention is a quantity which will result in a therapeutic or prophylactic benefit for the animal. The effective amount will vary, depending on such factors as the route of administration, the condition of the patient, the nature and extent of the disease's effects, and the like. Such factors are capable of determination by those skilled in the art.

As used herein, the term “effective amount” also means the total amount of each active component of the composition or method that is sufficient to show a meaningful patient benefit, i.e., treatment, healing, prevention or amelioration of the relevant medical condition, or an increase in rate of treatment, healing, prevention or amelioration of such conditions. For example, an effective amount of a compound is an amount sufficient to achieve a desired therapeutic and/or prophylactic effect, e.g., to thereby treat an inflammatory disorder or symptom thereof. When applied to a combination, the term refers to combined amounts of the active ingredients that result in the therapeutic effect, whether administered in combination, serially or simultaneously.

For example, for a subject with rheumatoid arthritis, a “beneficial clinical outcome” compared with the absence of the treatment includes a reduction in the severity of the symptoms associated with the inflammation, e.g., pain, swelling, fever, rash, and the like, a reduction in the rate of tissue or bone degeneration, an increase in the range of motion of an affected joint in a subject, a reduction in the rate of decrease in the range of motion of an affected joint in a subject, an increase in the longevity of the subject, and the like.

The precise amount of compound administered to a subject will depend on the type and severity of the disease or condition and on the characteristics of the subject, such as general health, age, sex, body weight and tolerance to drugs. It will also depend on the degree, severity and type of inflammatory disorder. The skilled artisan will be able to determine appropriate dosages depending on these and other factors.

The OSM or IL-1 signaling inhibitors described herein and the compounds of the present invention and additional therapeutic agents described herein can be administered to a subject by any conventional method of drug administration, for example, orally in capsules, suspensions or tablets or by parenteral administration. Parenteral administration can include, for example, intramuscular, intravenous, intraventricular, intraarterial, intrathecal, subcutaneous, or intraperitoneal administration. The OSM or IL-1 signaling inhibitors described herein and the compounds of the present invention or agent can also be administered orally (e.g., in capsules, suspensions, tablets or dietary), nasally (e.g., solution, suspension), transdermally, intradermally, topically (e.g., cream, ointment), inhalation (e.g., intrabronchial, intranasal, oral inhalation or intranasal drops) transmucosally or rectally. Delivery can also be by injection into the brain or body cavity of a patient or by use of a timed release or sustained release matrix delivery systems, or by onsite delivery using micelles, gels and liposomes. Nebulizing devices, powder inhalers, and aerosolized solutions may also be used to administer such preparations to the respiratory tract. Delivery can be in vivo, or ex vivo. Administration can be local or systemic as indicated. More than one route can be used concurrently, if desired. The preferred mode of administration can vary depending upon the particular agent chosen.

In specific embodiments, oral, parenteral, or system administration are preferred modes of administration for treatment of inflammatory disorders.

The OSM or IL-1 signaling inhibitors described herein and the compounds of the present invention can be administered alone as a monotherapy, or in conjunction with one or more additional therapeutic agents. The term “in conjunction with,” indicates that the compound is administered at about the same time as the agent. The compound can be administered to the animal as part of a pharmaceutical composition comprising the compound and a pharmaceutically acceptable carrier or excipient and, optionally, one or more additional therapeutic agents. The compound and compound can be components of separate pharmaceutical compositions which can be mixed together prior to administration or administered separately. The compound can, for example, be administered in a composition containing the additional therapeutic agent, and thereby, administered contemporaneously with the agent. Alternatively, the compound can be administered contemporaneously, without mixing (e.g., by delivery of the compound on the intravenous line by which the compound is also administered, or vice versa). In another embodiment, the compound can be administered separately (e.g., not admixed), but within a short time frame (e.g., within 24 hours) of administration of the compound.

Additional therapeutic agents which can be coadministered with the OSM or IL-1 signaling inhibitors described herein and the compounds of the present invention include, but are not limited to, drugs (including DMARDS)methorexate, monoclonal or murine, chimeric, human or humanized antibodies, fragments and regions thereof, and the like.

The preparation of a pharmacological composition that contains active ingredients dissolved or dispersed therein is well understood in the art. Typically such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

Also contemplated within the invention are compositions and kits comprising at least one OSM or IL-1 signaling inhibitors described herein or the compounds of the present invention. The compositions and kits may optionally contain one or more additional therapeutic agents.

The methods of the present invention contemplate single as well as multiple administrations, given either simultaneously or over an extended period of time. The compound (or composition containing the compound) can be administered at regular intervals, depending on the nature and extent of the inflammatory disorder's effects, and on an ongoing basis. Administration at a “regular interval,” as used herein, indicates that the therapeutically effective amount is administered periodically (as distinguished from a one-time dose). In one embodiment, the compound is administered periodically, i.e., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased. Depending upon the half-life of the agent in the particular animal or human, the agent can be administered between, for example, once a day or once a week.

For example, the administration of the OSM or IL-1 signaling inhibitors described herein or the compounds of the present invention and/or the additional therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g, between 6:00 a.m. and 12:00 noon; in the afternoon, e.g., after noon and before 6:00 p.m.; or in the evening, e.g., between 6:01 p.m. and midnight. The compound can be administered before, during or after the onset of the inflammatory disorder.

The OSM or IL-1 signaling inhibitors described herein or the compounds of the present invention and/or additional therapeutic agent can be administered in a dosage of, for example, 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day. Dosage forms (composition) suitable for internal administration generally contain from about 0.1 milligram to about 500 milligrams of active ingredient per unit. In these pharmaceutical compositions the active ingredient will ordinarily be present in an amount of about 0.5-95% by weight based on the total weight of the composition.

The amount of compound or agent administered to the individual will depend on the characteristics of the individual, such as general health, age, sex, body weight and tolerance to drugs as well as the degree, severity and type of rejection. The skilled artisan will be able to determine appropriate dosages depending on these and other factors using standard clinical techniques.

In addition, in vitro or in vivo assays may optionally be employed to help to identify optimal dosage ranges. The precise dose to be employed will also depend on the route of administration, the seriousness of the disease, and the individual's circumstances. Effective doses may be extrapolated from dose-response curves derived from in vitro or animal model test systems. The amount of the compound will also depend on the disease state or condition being treated along with the clinical factors and the route of administration of the compound.

For treating humans or animals, about 1 mg/kg of body weight to about 20 mg/kg of body weight of the OSM or IL-1 signaling inhibitors described herein or the compounds of the present invention can be administered. In a preferred embodiment, the effective amount of agent or compound is about 1-10 mg/kg body weight of the individual. In another embodiment, the effective amount of agent or compound is about 1-5 mg/kg body weight of the individual. The effective amount for a particular individual can be varied (e.g., increased or decreased) over time, depending on the needs of the individual.

The term “unit dose” when used in reference to a therapeutic composition of the present invention refers to a physically discrete unit suitable as unitary dosage for the subject, each unit containing a predetermined quantity of active material calculated to produce the desired therapeutic effect in association with the required diluent; i.e., carrier or vehicle. In addition to the ingredients particularly mentioned above, the formulations of the present invention may include other agents conventional in the art having regard to the type of formulation in question.

The OSM or IL-1 signaling inhibitors described herein and the compounds of the present invention described herein can be administered to the subject in conjunction with an acceptable pharmaceutical carrier or diluent as part of a pharmaceutical composition for therapy. Formulation of the compound to be administered will vary according to the route of administration selected (e.g., solution, emulsion, capsule, and the like). Suitable pharmaceutically acceptable carriers may contain inert ingredients which do not unduly inhibit the biological activity of the compounds. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

As used herein, the term “pharmaceutically acceptable”, means that the materials (e.g., compositions, carriers, diluents, reagents, salts, and the like) are capable of administration to or upon a mammal with a minimum of undesirable physiological effects such as nausea, dizziness or gastric upset.

In one embodiment, the method comprises topical administration. In such cases, the compounds may be formulated as a solution, gel, lotion, cream or ointment in a pharmaceutically acceptable form. Actual methods for preparing these, and other, topical pharmaceutical compositions are known or apparent to those skilled in the art and are described in detail in, for example, Remington's Pharmaceutical Sciences, 16^{th} and 18^{th} eds., (Mack Publishing Company, Easton, Pa., 1980-1990).

Also included in the present invention are pharmaceutically acceptable salts of the OSM or IL-1 signaling inhibitors described herein and the compounds of the present invention described herein. These OSM or IL-1 signaling inhibitors described herein and the compounds of the present invention can have one or more sufficiently acidic protons that can react with a suitable organic or inorganic base to form a base addition salt. When it is stated that a compound has a hydrogen atom bonded to an oxygen, nitrogen, or sulfur atom, it is contemplated that the compound also includes salts thereof where this hydrogen atom has been reacted with a suitable organic or inorganic base to form a base addition salt. Base addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Such bases useful in preparing the salts of this invention thus include sodium hydroxide, potassium hydroxide, ammonium hydroxide, potassium carbonate, and the like.

For example, pharmaceutically acceptable salts of the OSM or IL-1 signaling inhibitors described herein and the compounds of the present invention are those formed by the reaction of the OSM or IL-1 signaling inhibitors described herein or the compounds of the present invention with one equivalent of a suitable base to form a monovalent salt (i.e., the compound has single negative charge that is balanced by a pharmaceutically acceptable counter cation, e.g., a monovalent cation) or with two equivalents of a suitable base to form a divalent salt (e.g., the compound has a two-electron negative charge that is balanced by two pharmaceutically acceptable counter cations, e.g., two pharmaceutically acceptable monovalent cations or a single pharmaceutically acceptable divalent cation). “Pharmaceutically acceptable” means that the cation is suitable for administration to a subject. Examples include Li⁺, Na⁺, K⁺, Mg²⁺, Ca²⁺ and NR₄⁺, wherein each R is independently hydrogen, an optionally substituted aliphatic group (e.g., a hydroxyalkyl group, aminoalkyl group or ammoniumalkyl group) or optionally substituted aryl group, or two R groups, taken together, form an optionally substituted non-aromatic heterocyclic ring optionally fused to an aromatic ring. Generally, the pharmaceutically acceptable cation is Li⁺, Na⁺, K⁺, NH₃(C₂H₅OH)⁺ or N(CH₃)₃(C₂H₅OH)⁺.

OSM or IL-1 signaling inhibitors described herein or the compounds of the present invention with a sufficiently basic group, such as an amine, can react with an organic or inorganic acid to form an acid addition salt. Acids commonly employed to form acid addition salts from compounds with basic groups are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

It will also be understood that certain OSM or IL-1 signaling inhibitors described herein and the compounds of the present invention may be obtained as different stereoisomers (e.g., diastereomers and enantiomers) and that the invention includes all isomeric forms and racemic mixtures of the disclosed compounds and methods of treating a subject with both pure isomers and mixtures thereof, including racemic mixtures. Stereoisomers can be separated and isolated using any suitable method, such as chromatography.

### EXEMPLIFICATION

### Example 1

### SENTINEL® Primary Cell Line Screen Identifies OSM Signaling Inhibitors

A SENTINEL® cell line (Bionaut, Cambridge, Mass.) was established to identify small molecules that inhibit OSM signal transduction. Cell line 4A1 was established in A549 cells (a human lung cancer cell line) using BV32, a vector that carries both the luciferase and GFP reporters. Assay development was performed to optimize the conditions of the OSM-response in the 4A1 SENTINEL® line. This assay reached a Z′ of 0.7, a desirable value that indicates excellent well-to-well uniformity.

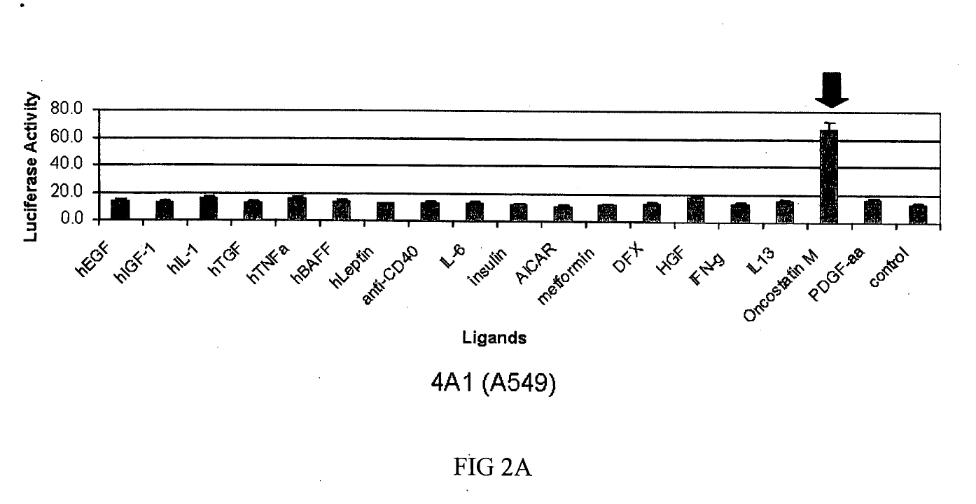

FIGS. 2A-E are charts showing that 4A1 responds specifically to OSM stimulation in a time- and dose-dependent manner.

FIG. 2A is a bar graph showing the specific response of the 4A1 cell line to OSM as measured by luciferase reporter activity in comparison to a panel of 17 other ligands.

FIGS. 2B-C are log-log plots showing that luciferase reporter activation (Y-axis) was corroborated by green fluorescent protein (GFP) reporter (X-axis) by fluorescence-activated cell sorting (FACS) analysis.

FIG. 2D-E are a pair of graphs showing time- and dose-dependent activation of reporter activity by OSM. FIG. 2D shows the increase in reporter activity versus time at an OSM concentration of 10 nanograms/milliliter (ng/mL). FIG. 2E shows the response (in luciferase/10,000 cells) versus OSM concentration in ng/mL.

Approximately 1800 compounds were screened, from which 100 compounds (5.6%) were identified that inhibited the OSM-stimulated (luciferase) reporter activity by ≧50% in 4A1. 18 compounds passed retests, and 11 produced desirable dose response curves using reordered dry compounds. Table 1 shows the identified OSM inhibitors.

TABLE 1
Primary screen

| Compound | IC₅₀ |
|---|---|
| 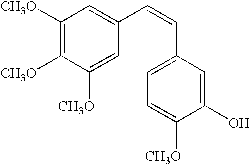 | 0.003 |
| Combretastatin A4 (1) | |
| 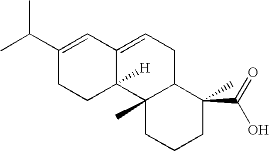 | 0.012 |
| Abietic acid (2) | |
| 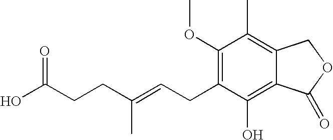 | 0.02 |
| Mycophenolic acid (3) | |
| 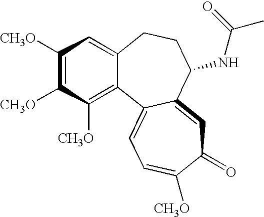 | 0.1 |
| Colchicine (4) | |
| 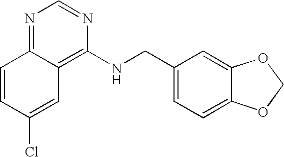 | 0.4 |
| MBCQ (5) | |
| 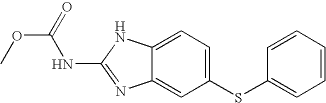 | 0.8 |
| Fenbendazole (6) | |
| 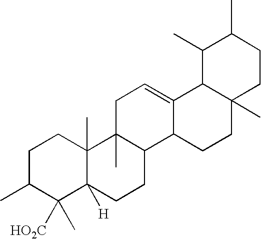 | 1.9 |
| β-boswellic acid (7) | |
| 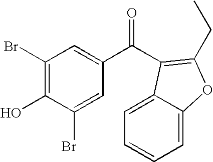 | 2.2 |
| Benzobromarone (8) | |
| 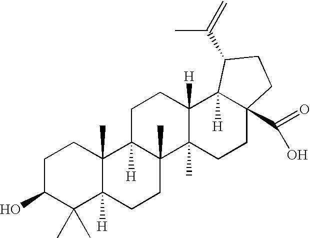 | 3.3 |
| Betulinic acid (9) | |
| 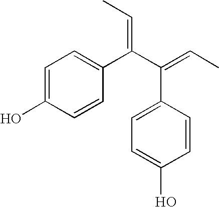 | 3.5 |
| Dienestrol (10) | |
| 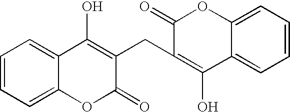 | 3.7 |
| Dicumarol (11) | |

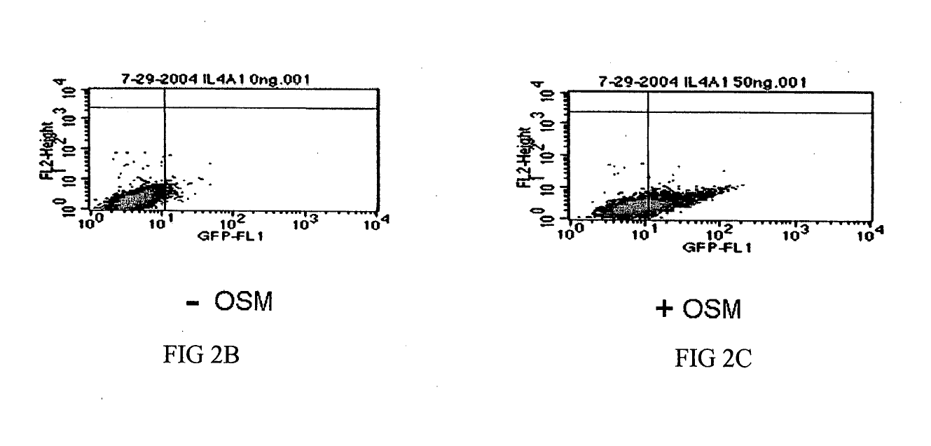

FIGS. 3A-B demonstrate the inhibitory activity of OSM signaling inhibitors (1)-(11) in the 4A1 cell line. Each IC₅₀ value is the (micromolar, μM) concentration of OSM signaling inhibitor that inhibits OSM-stimulated (luciferase) reporter activity by ≧50%. FIG. 3A is a graph showing the concentration of OSM signaling inhibitors (1) and (11) versus normalized (luciferase) reporter activity. FIG. 3B is a table of IC₅₀ values for OSM signaling inhibitors (1)-(11).

### Example 2

### Cartilage Cell Line Screen Verifies OSM Signaling Inhibitors

To test the compounds identified in the preliminary reporter (luciferase) activity SENTINEL® line 4A1 screen for inhibition of OSM signaling in a more physiologically relevant environment, a human cell line involved in an inflammatory disease was adapted. The human cartilage cell line SW-1353 was selected.

In RA, connective tissue destruction can be primarily mediated by chondrocytes, synovial fibroblasts, and, on occasion, by osteoclasts (M. P. Vincenti, C. E. Brinckerhoff, Arthritis Res 4, 157 (2002)). The interstitial collagens (type I, II, III) can be the principal targets of destruction, and the secreted collagenases, which can digest and unwind the triple helix of collagen to make it available to attack by other proteinases, are believed to play a role in collagen degradation and cartilage destruction. Among these collagenases, MMP-13 may have a particular role in cartilage degradation because it has a more restrictive expression within chondrocytes and bone, and because it hydrolyzes type II collagen more efficiently than the other collagenases (P. G. Mitchell et al., J Clin Invest 97, 761 (Feb. 1, 1996)). Moreover, expression of MMP-13, but not of MMP-1 (another collagenase), is believed to be regulated in the same manner in response to IL-1 stimulation in both human cartilage cells and primary chondrocytes (J. A. Mengshol, M. P. Vincenti, C. E. Brinckerhoff, Nucleic Acids Res 29, 4361 (Nov. 1, 2001)).

In the human cartilage cell line SW-1353, while IL-1 is believed to activate expression of collagenases to a small degree and OSM is believed to have no effect on collagenase expression, a combination of IL-1 and OSM is believed to lead to a synergistic activation of collagenases to much higher levels (S. Cowell et al., Biochem J 331 (Pt 2), 453 (Apr. 15, 1998). Regulation of MMP-13 in SW-1353 cells is, therefore, believed to provide a biomarker which can be used to monitor IL-1/OSM-mediated signal transduction events in secondary assays.

MMP-2 production can be used as control for the potential side-effects of compounds. This is because MMP-2 levels are not believed to change in response to IL-1/OSM.

The same supernatants of culture media used in the primary screen were assayed for IL-1/OSM-stimulated pro-MMP-13 production and MMP-2 production by enzyme-linked immunosorbent assay (ELISA). The IC₅₀ values of inhibition of pro-MMP-13 production ranged from 2 nM to 32 μM in this experiment.

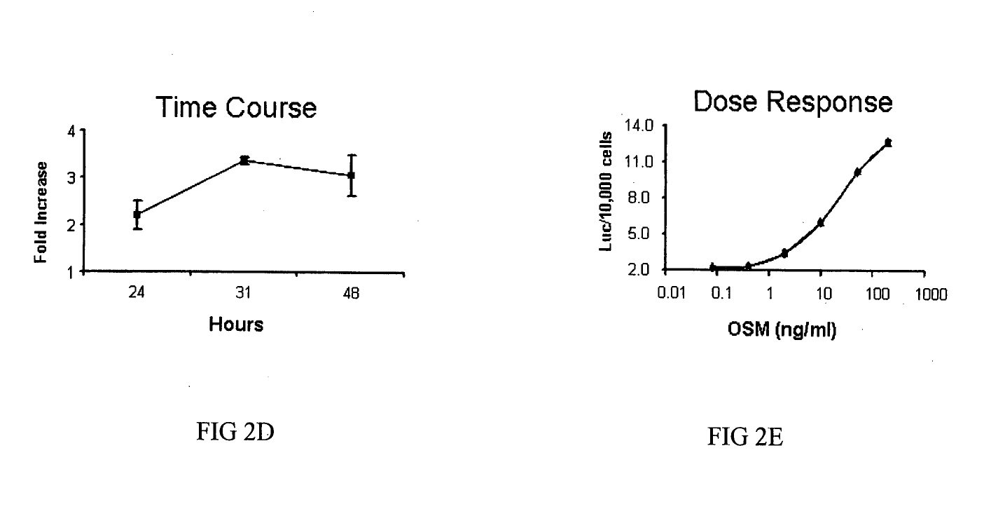

FIGS. 4A-B demonstrate the inhibitory activity of OSM signaling inhibitors (1), (2), (8), (9), and (12) against MMP-13 production a secondary screen in chondrocyte cell line SW-1353. FIG. 4A is a graph showing the concentration of OSM signaling inhibitor (μM) versus MMP-13 expression (picograms/10,000 cells). FIG. 4B is a table of IC₅₀ values for MMP-13 inhibition by OSM signaling inhibitors (1), (2), (8), (9), and (12).

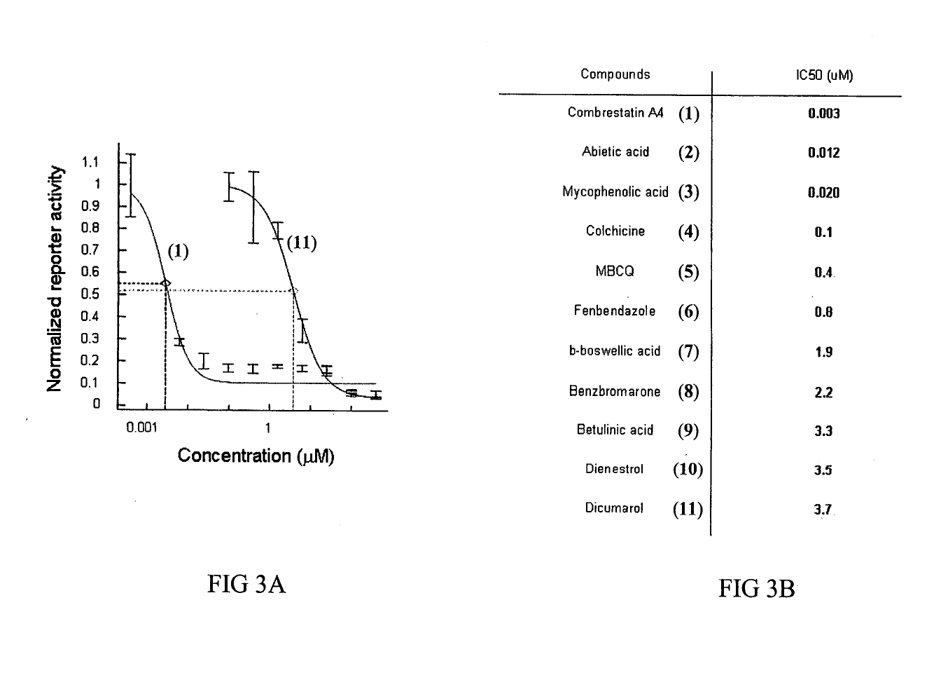

The compounds that showed dose-dependent activity in this MMP-13 secondary assay are combretastatin A-4 (CA4) (OSM signaling inhibitor 1) (IC₅₀=2 nM), abietic acid (OSM signaling inhibitor 2) (6 nM), benzobromarone (OSM signaling inhibitor 8) (0.4 μM), betulinic acid (OSM signaling inhibitor 9) (0.6 μM), and pifithrine-α (OSM signaling inhibitor 12) (32 μM). Colchicine (OSM signaling inhibitor 4) at 4 μM resulted in approximately 83% reduction of signal. In contrast, there was no or only modest inhibition of MMP-2 production when concentrations ≧IC₅₀ of the compounds were used, indicating that these compounds inhibited MMP-13 production more dramatically than MMP-2 production. FIG. 5 is a series of three bar graphs that show OSM signaling inhibitors (1), (2), (8), (9), and (12) having greater inhibitory effects on MMP-13 expression compared to MMP-2 expression.

Selected compounds were tested in the secondary screen. Table 2 shows the IC₅₀ (μM) results of the secondary screen.

TABLE 2
Secondary Screen

| Compound | IC₅₀ |
|---|---|
| 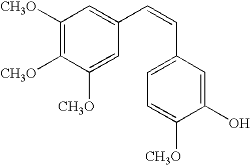 | 0.002 |
| Combretastatin A4 (1) | |
| 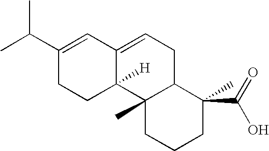 | 0.006 |
| Abietic acid (2) | |
| 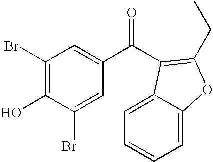 | 0.4 |
| Benzobromarone (8) | |
| 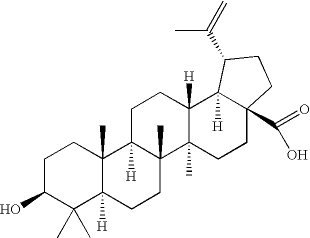 | 0.6 |
| Betulinic acid (9) | |
| 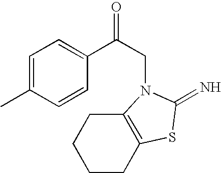 | 32 |
| Pifithrine-alpha (12) | |

### Example 3

### In Vivo Studies Validate Efficacy of OSM Signaling Inhibitor (1)

OSM signaling inhibitor (1) (CA4) can be tested in a rodent arthritis model to investigate in vivo efficacy. The collagen-induced arthritis (CIA) model is chosen due to its pathohistological resemblance to human RA. CIA is also the industry standard animal model to test DMARDs, in particular those that target cytokines.

OSM signaling inhibitor (1) is solublized at 6-12 mM in 2-4% NMP/2-4% solutol/2% methocel. The compound is administered subcutaneously via osmotic pumps to provide a steady infusion of the compound. This may be necessary for OSM signaling inhibitor (1) in that it has a short half time (approximately 0.5 hour) in mouse plasma (I. G. Kirwan et al., Clin Cancer Res 10, 1446 (Feb. 15, 2004)). As a preliminary step, in vitro tests to verify OSM signaling inhibitor (1) release from the pump and stability studies during a planned course of in vivo infusion (14 days) are conducted. From the data, a maximum tolerable dose (MTD) and in vivo bioavailability study are performed to determine an optimal dose range. A 4-arm in vivo study of the CIA model is conducted that can include a disease control, vehicle only, vehicle+OSM signaling inhibitor (1), and a positive control.

### Example 4

### Mechanism of Action (MOA) Study for OSM Signaling Inhibitors

The mechanism of action of the OSM signaling inhibitors can be investigated. For example, for OSM Signaling Inhibitor (1), signaling pathways that may be critical to the observed inhibition of MMP-13 production are mapped. Candidate pathways can include JAK/STAT, JNK, ERK, p38, NFkB, and RUNX. A combination of pharmacological and biological inhibitors of known signaling pathways and promoter reporters that respond specifically to these pathways are employed. For any lead candidates of which the molecular targets are unknown, labeling technology can be employed to identify these targets.

### Example 5

### Cell Line Screens

Methods

Cell Culture and Reagents—A549 human lung carcinoma and SW1353 human chondrosarcoma cell lines were obtained from the American Type Culture collection, and were maintained in Dubecco's modified Eagle's medium (DMEM), and DMEM plus Ham's F12, respectively, with 10% fetal-bovine serum (FBS) in a humid atmosphere containing 5% CO₂ at 37° C. Oncostatin M (OSM), and IL-1β, were from Peprotech (Rocky Hill, N.J.). JAK inhibitor I, SB203580, PD98059, Kamebakaurin, and SP600125 were from Calbiochem (San Diego, Calif.). All phopho-specific antibodies were from BD Biosciences (San Diego, Calif.): Stat-1 (pY701), Stat3 (pY705), Stat5 (Y694), p38 (pT180/pY182), JNK (pT183/pY185), and ERK1/2 (pT202/pY204).

Generation of Sentinel Clones

The Sentinel clones were generated as described in US patent application (US20020076688A1) the entire contents of which are incorporated herein by reference.

Luciferase Reporter Assays and Library Screening

Reporter activities of Sentinel® clones were measured using the Steady-Glo® Luciferase assay system (Promega, Madison, Wis.), according to the manufacturer's instructions, and were normalized to cell numbers based on nucleic acid quantification (CyQuant® cell proliferations assay, Molecular Probes/Invitrogen, Carlsbad, Calif.). In order to identify potential anti-inflammatory compounds, the OSM-responding Sentinel® clone 4A1 was used for a library screen of approximately 1800 compounds.

Measurement of MMPs

For matrix metalloproteinase (MMP) measures, SW1353 cells were grown to confluence in 24 well plates, washed in DMEM (with Ham's F12) and placed in 500 ul of serum free DMEM/F-12 with 0.2% lactalbumin hydrosylate with and without OsM/IL-1. After 24 hrs, supernatants were collected and the amounts of proMMP13, totalMMP2, and proMMP1 were measured using Quantikine® ELISA kit for human MMPs (R & D systems, Minneapolis, Minn.). All MMP measures were normalized to viable cell numbers based on a tetrazolium (MTS) colorimetric measure (Promega, Madison, Wis.).

Intracellular-Phospho-Specific Flow Cytometry and Immunohistochemistry

SW1353 cells were seeded and treated as described above. For the flow cytometric analysis of intracellular phopho-proteins, trypsinized SW1353 cells were fixed in 1.5% formaldehyde at room temperature (RT), and permeabilized in ice cold methanol for minimum of 10 minutes. After 2× wash in wash buffer (D-PBS, with 1% FBS), cells were stained with phospho-specific antibodies (5 ug/ml final) for 20 minutes, washed 2× in wash buffer, followed by 20 minutes incubation with phycoerythrin-conjugated anti-mouse IgG (10 ug/ml final). Following 2× wash in wash buffer, cells were analyzed by flow cytometry.

For immunohistochemistry of intracellular phospho-proteins, fixation and permeabilization conditions were identical to the ones used for flow cytometric analysis. For the visualization of the phosphor-specific proteins, ImmPRESS Universal Antibody kit (Vector Laboratories, Burlingame, Calif.) was used.

SW1353 Cell Transfection

SW1353 cells were plated at 100,000 in 24-well plates and cultured overnight in 10% FCS-DMEM/F-12. The following day, cells were transfected with 0.3 ug total DNA (pNF-kB-Luc reporter plasmid, BD biosciences San Jose, Calif.) using Lipofectamine™ 2000 (Invitrogen), according to the manufacturer's instructions. After overnight incubation, cells were washed in F12 media, pretreated with CA4 for 2 hrs, and then activated with IL-1/OSM (6 hrs) before measuring reporter activity.

Taqman

Total RNA was isolated using RNAzol™ B (TEL-TEST Inc., Friendswood, Tex.). SuperScript™ first-strand synthesis system (Invitrogen) was used for cDNA generation. All oligonucleotide primers and fluorescent-labeled TaqMan probes were from Applied Biosystems (Foster City, Calif.). Relative quantization of gene expression was performed using the MX3000p real-time PCR instrument (Stratagene, La Jolla, Calif.). PCR reactions for all samples were performed in triplicates using 50 ng of cDNA. To compensate for the variation in the total cDNA, all readouts were normalized to an endogenous control (18S ribosomal RNA). Thermocycler conditions comprised an initial holding at 50° C. for 2 min, then 95° C. for 10 min, followed by a 2-step TaqMan PCR program consisting of 95° C. for 15 seconds, and 60° C. for 60 seconds for 40 cycles. The relative changes in gene expression were quantified as described in Applied Biosystems User Bulletin NO. 2 (P/N 4303859).

Statistical Analysis and Curve Fitting

Regression lines were plotted using XLfit4 (ID Business Solutions Inc., Cambridge, Mass.) with four parameter logistic curve fitting. All data are reported as mean±SEM. P values (Student t-test, paired) less than 0.05 were considered significantly different.

### Example 6

### Identification and Characterization of a Sentinel® Cell Line that Responds to OSM and IL-1

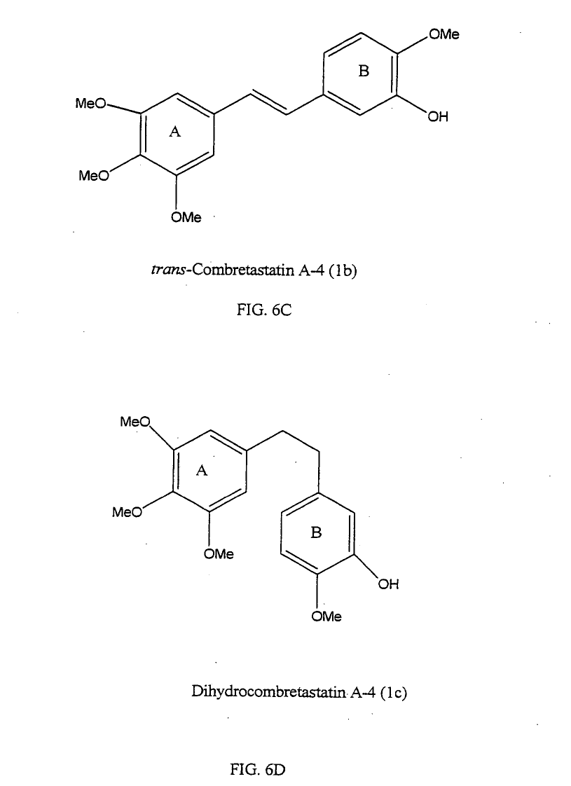

Sentinel lines were developed by utilizing promoter trapping technology in combination with positive and negative selections to efficiently deliver reporter genes into endogenously regulated genetic sites. Using this approach, a Sentinel cell line, 4A1, in A549 lung carcinoma cells that responded specifically to OSM and IL-1 stimulation among a panel of cytokines and active compounds was established (FIG. 9A).

OSM dose-dependently activated the luciferase reporter activity of 4A1 5-7 folds over background with EC50 of approximately 50 ng/ml (FIGS. 9A and B). Interestingly, this activation appeared to be unique to OSM because the other members of the gp-130-binding cytokine family, IL-6, IL-11, Ciliary Neurotrophic Factor (CNTF), leukemia inhibitory factor (LIF) and cardiotrophin-1 (CT-1), had no stimulatory effect (data not shown). The 4A1 reporter activity was also activated by IL-1 with EC50 of 28 pg/ml (FIG. 9C). When OSM and IL-1 were added together to 4A1 cells, the reporter activity appeared to be moderately more than the sum of individual activities obtained when OSM and IL-1 added separately (FIG. 9D). However, the luciferase activity of 4A1 appeared to respond predominantly to OSM, because OSM typically produced 5-7-fold maximum activation whereas IL-1 only produced 2-fold maximum activation (FIGS. 9B and C).

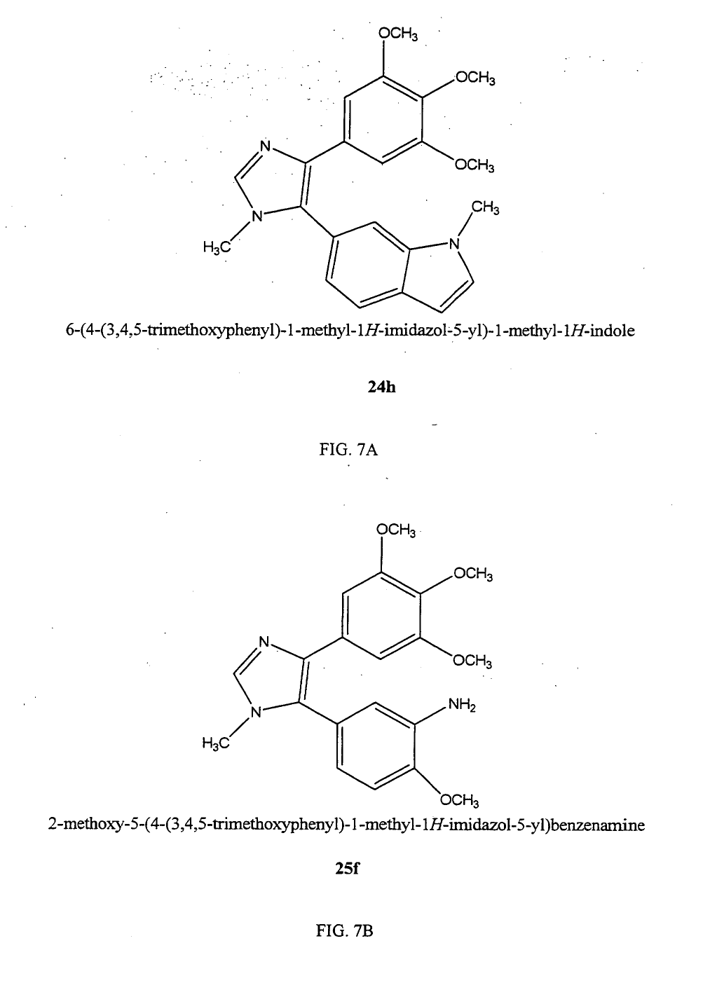

To verify that the increase of 4A1 luciferase activity in the presence of OSM was a result of OSM signaling, the effects of inhibitors that block the canonical OSM signal transduction pathways were tested. As shown in FIG. 10A-E, JAK inhibitor (JAK inhibitor I), MEK inhibitor (PD98059), p38 inhibitor (SB203580), JNK inhibitor (SP600125), and NF-κB inhibitor (Kamebakaurin, KA) inhibited the reporter activity in a dose-dependent manner, which is consistent with published results Grant et al., Molecular Medicines Today 5, 406412 (1999), Godoy-Tundidor et al., Prostate 64, 209-216 (2005), Nishibe et al., Blood, 97, 692-699 (2001), Tamura et al., Neuroscience, (2005) 133 (3), 797-806, 133(3) 615-24, 130(1) 233-8, Weiss et al., J Mol Cell Cardiol, (2005) 39(3), 545-51, Li et al., J Immunol 166, 3491-3498 (2001). Wang et al., J. Biol. Chem. 275 25273-25285 (2000) the entire contents of each of which are incorporated herein by reference. These results suggested that OSM activated cellular signal transduction pathways that led to increased reporter activity. Among them, JAK/STAT, ERK, p38, JNK, and NF-κB pathways all appeared to be important for reporter activation in 4A1 cells.

### Example 7

### Combretastatin A-4 Inhibits OSM- and IL-1-Stimulated Reporter Activity

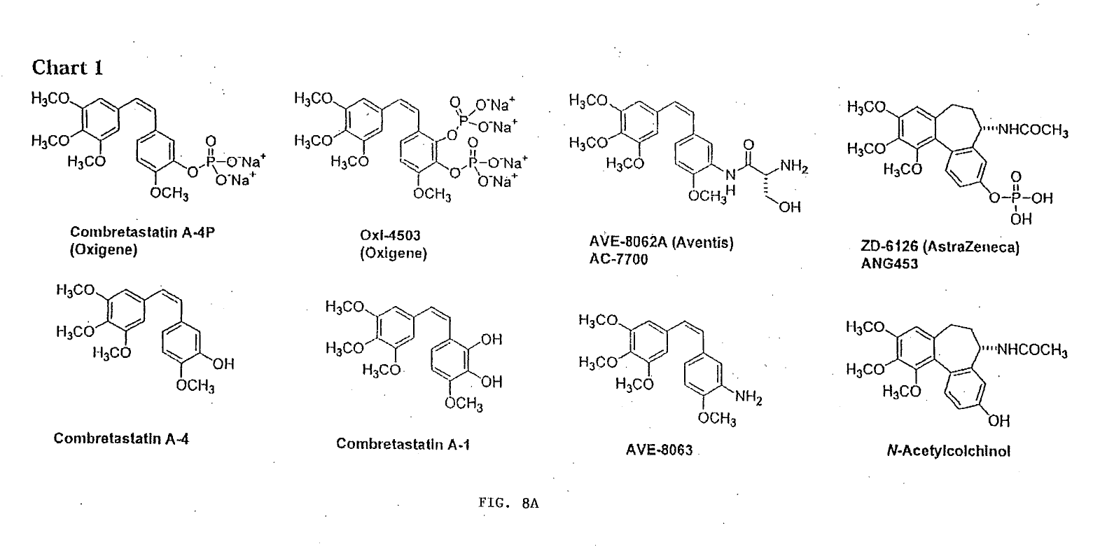

To identify compounds that inhibit OSM signaling, the collection of approximately 1800 compounds was screened using the 4A1 Sentinel line. Combretastatin A-4 (CA4), potently inhibited the reporter activity in response to both OSM and IL-1 ligands, with IC50's for OSM- and IL-1-stimulated reporter activities at 5.3±0.5 nM and 6.9±0.4 nM, respectively (FIGS. 11A, B).

### Example 8

### Combretastatin A-4 Inhibits OSM- and IL-1-Stimulated Expression of MMP-13 in Chondrosarcoma Cells

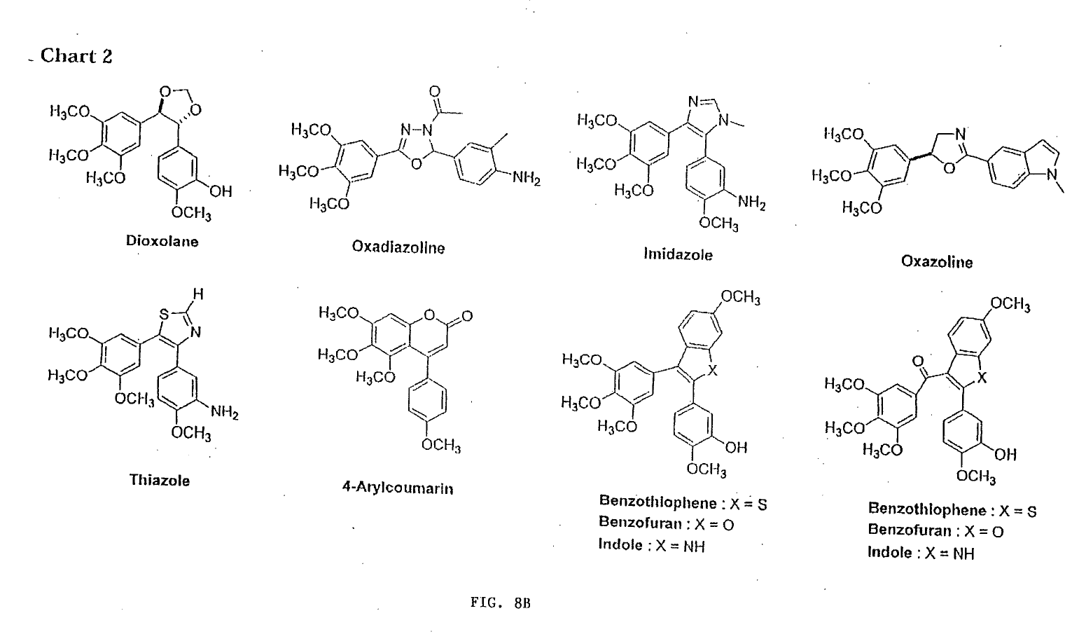

The human chondrosarcoma cell line, SW1353, was used as the cell system for examining CA4's effect on OSM- and IL-1-mediated signal transduction. Collagenase-3 (MMP-13) is restrictively expressed in cartilage (Mitchell et al. J Clin Incest 97, 761-768 (1996) and Vincenti et al., Biochem J 331 (Pt 1) 341-346 (1998), the entire contents of which are incorporated herein by reference), and is upregulated by OSM and IL-1 stimulation in cultured and primary chondrocytes (Koshy et al. Ann Rheum Dis 61, 704-713 (2002), Li et al., J Immunol 166 3491-3498 (2001), Cowell et al., Biochem J 331 453-458 (1998), Mengshol et al., Nucleic Acids Res 29 4361-4372 (2001) and Mengshol et al., Arthritis Rheum 43, 801-811 (2000) the entire contents of each of which are incorporated herein by reference). Therefore, MMP-13 was used as a surrogate marker of CA4 effects on OSM and IL-1 signaling in SW1353 cells. As shown in FIG. 12A, CA4 dose-dependently inhibited OSM/IL-1-mediated production of pro-MMP-13 with an IC50 of 5.6±1.3 nM. Consistent with its effect on IL-1 mediated reporter activity, CA4 also inhibited IL-1-stimulated pro-MMP-13 production with an IC50 of 5.2±1.9 nM (FIG. 12B), despite much weaker stimulation by IL-1 alone (FIG. 12). These results suggest that CA4 blocks the synergistic signal transduction by OSM and IL-1, as well as the signal transduction by IL-1 alone, in SW1353 cells. This observation is in agreement with that obtained with the 4A1 Sentinel® reporter line.

Although OSM synergized with IL-1 to activate MMP-13 transcription in SW1353 cells (FIG. 12, (30, 74)), OSM alone did not stimulate MMP-13 expression in SW1353 cells (Cowell et al., Biochem J 331 453-458 (1998) the entire contents of which are incorporated herein by reference and data not shown). As a result, we could not test directly in SW1353 cells whether CA4 also blocked OSM signaling. However, in an OSM-responsive SW1353 Sentinel cell line, CA4 dose-dependently inhibited the reporter luciferase activity (FIG. 12C), suggesting that CA4 could disrupt OSM signaling in SW1353 cells. Further, when dedifferentiated primary chondrocytes were tested, CA4 also inhibited OSM-stimulated pro-MMP-13 production dose-dependently (FIG. 12D). Taken together, it appears that CA4 interferes as well with OSM signal transduction that led to the synergistic upregulation of pro-MMP-13 by OSM/IL-1 in SW1353 cells.

### Example 9

### Combretastatin A-4 Fails to Inhibit MMP-2 Expression and TGF-β- and CD3-Mediated Sentinel Reporter Activities

Multiple control assays were performed to investigate the selectivity of CA4 action. Matrix metalloproteinase-2 (MMP-2) production in SW1353 cells was tested. Mutation of MMP-2 causes a multicentric osteolysis and arthritis syndrome (Martignetti et al., Nat Genet 28, 261-265 (2001) the entire contents of which are incorporated herein by reference), and theoretically its expression should not be affected in treating RA. CA4 at approximately 110 nM had no effect on production of MMP-2 (FIG. 12E). An A549 Sentinel line that responded to TGF-β was then tested. An inhibitor of TGF-β receptor (SB431542) was able to inhibit the TGF-β-induced reporter activity of this Sentinel® line. However, CA4 as high as 1 μM had no effect on TGF-β-stimulated reporter activity, suggesting that not all signal transduction pathways were affected by CA4 (data not shown). Finally, CA4 was tested against an unrelated GFP-reporter Jurkat T-cell line whose reporter activity can be upregulated by an anti-CD3 antibody (unpublished data). CA4 at 20 nM did not have any effect on the CD3-mediated increase of GFP fluorescence (data not shown). Taken together, our data suggest that CA4 did not disrupt the general transcriptional, translational, or protein trafficking machinery, and that CA4 actions were selective.

### Example 10

### Inhibition by MIAs of OSM-MEDIATED Reporter Activity and MMP-13 Expression

Other microtubule interfering agents (MIAs) were also tested for inhibition of OSM-mediated reporter activity in 4A1 and OSM/IL-1-mediated pro-MMP-13 production in SW1353 cells. As illustrated in Table 3, vinblastine and CA4 were the most potent MIAs in inhibition of the reporter activity with IC50s of 3.4±0.6 nM and 5.3±0.5 nM, respectively, and CA4 and vinblastine were the most potent in inhibition of pro-MMP-13 production with IC50s of 5.6±1.3 nM and 11.6±5.2 nM, respectively. Taxol, which stabilizes microtubule polymerization, was also tested on 4A1 reporter activity and MMP-13 production. Taxol dose-dependently inhibited 4A1 reporter activity under 200 nM, but had no effect on MMP-13 production (data not shown). In contrast to the above agents that interfere with microtubule dynamics, agents that disrupt actin filaments, such as cytochalacin D and cytochalacin B up to 1.5 μM, did not have effect on either the OSM-mediated reporter activity in 4A1 cells or OSM/IL-1-mediated pro-MMP-13 production in SW1353 cells (data not shown).

TABLE 3

| | | | | |
|---|---|---|---|---|
| CA-4 | Colchicine | Vinbalstine | Podophyllotoxin | Nocodazole |

IC50 Values by Luciferase Reporter Activity

| | | | | | |
|---|---|---|---|---|---|
| mean | 5.3 | 260.5 | 3.4 | 277 | 104 |
| SEM | 0.5 | 163.5 | 0.6 | 49 | 29.8 |

IC50 values by pro-MMP-13

| | | | | | |
|---|---|---|---|---|---|
| mean | 5.6 | 295 | 11.6 | 54.9 | >400 |
| SEM | 1.3 | 195 | 5.2 | 2.2 | N/A |

### Example 11

### Combretastatin A-4 Does not Affect JAK/STAT or MAP Kinase Pathways in SW1353 Cells

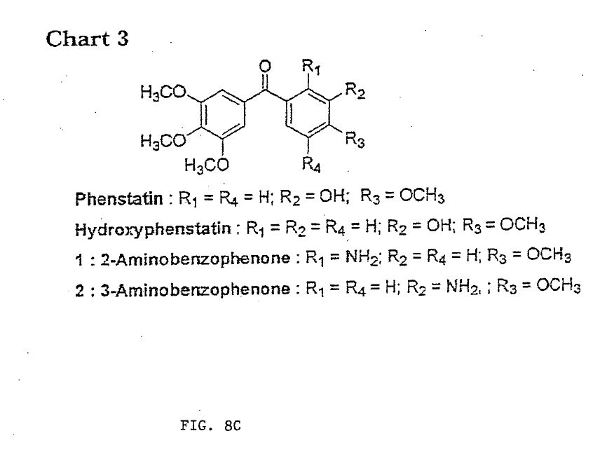

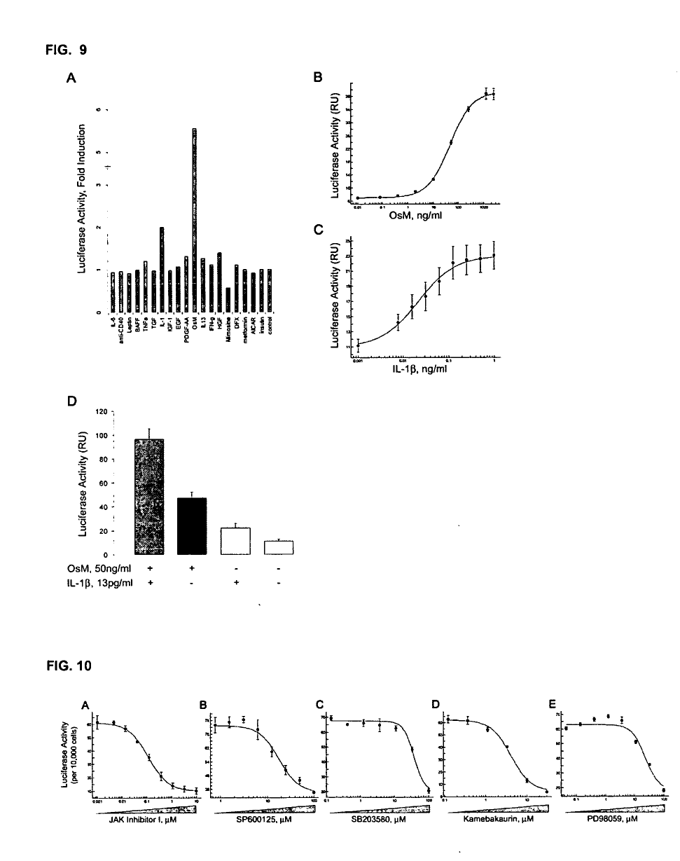

The cellular and molecular mechanisms by which CA4 repressed pro-MMP-13 production, using SW1353 as the model system were investigated. First it was demonstrated that OSM/IL-1 induced pro-MMP-13 expression was regulated at transcriptional level. Real time PCR (Taqman) showed a dramatic upregulation of MMP-13 mRNA upon OSM/IL-1 stimulation in SW1353 cells (FIG. 13A). The pathways that mediate OSM and IL-1 signal transduction were then examined. Using inhibitors that target key components of the pathways, it was shown that JAK/STAT, p38, JNK, and NF-κB pathways were important for OSM/IL-1-stimulated pro-MMP-13 production (FIG. 13B-E), but not ERK or Cox1/2 pathways (data not shown). These pathways were then examined to ascertain if activities of key components of these pathways were affected by CA4. CA4 at 20 nM did not affect STAT 1, 3, 5 phosphorylation by FACS analysis (FIG. 14A and data not shown) or nuclear translocation by immunostaining upon OSM/IL-1 activation (FIG. 14C and data not shown). Cells were pretreated with CA4 (20 nM) for 2 hrs and then activated with IL-1/OSM for 15 minutes. JNK (pT183/pY185), and ERK1/2 (pT202/pY204) phosphorylation was also not affected by CA4 (data not shown). Nor was p38 or JNK phosphorylation affected by 20 nM CA4 by FACS (FIG. 14B, and data not shown) or immunostaining (data not shown). CA4 did not inhibit NF-κB reporter activity, as well (FIG. 14D). Cells were pre-treated with CA4 or kamebakaurin (KA), for 2 hrs prior to IL-1/OSM activation. FIG. 14 D shows representative results from multiple experiments. CA4 did not affect the nuclear translocation of phospho Stat1 (dark purple, DAB+Ni staining). Stat3 and Stat5 nuclear translocation was also not affected by CA4 (data not shown). Therefore, it seemed that at least signal transduction events proximal to these key components in the important JAK/STAT, p38, and NF-κB pathways were not altered by CA4 treatment. The key transcriptional factors that regulate pro-MMP-13 expression, AP-1 (c-fos, c-Jun) and Runx2 were then examined (Mengshol et al., Nucleic Acids Res 29 4361-4372 (2001) and Mengshol et al., Arthritis Rheum 43, 801-811 (2000) Wang et al, Osteoarthritis Cartilage 12, 963-973, Vincenti et al., Arthritis Res 4, 157-164 (2002), the entire contents of each of which are incorporated herein by reference. Messenger RNA levels of none of these factors as detected by Taqman were significantly altered by treatment 20 nM CA4 in OSM/IL-1-stimulated or unstimulated SW1353 cells at 2 hr or 14 hr time points. Therefore, it appears that the effect on CA4 on pro-MMP-13 production was not through regulating levels of these transcriptional factors.

The mechanism of action of other OSM signaling inhibitors can be similarly investigated.

### Example 12

### Combretastatin A-4 Ameliorates Development of Arthritis in a Murine CIA Model

Male 6-8 week old B10RIII (Jackson Labs) mice were immunized at day 0, followed by a booster immunization on day 15. Alzet osmotic pumps (1002, Durect, Cupertino, Calif.) containing vehicle (12.5% N-methyl-2-Pyrrolidone/29% Solutol HS15) or various concentrations of CA4 were implanted subcutaneously on day 12, and dexamethasone was administered once daily subcutaneously at 0.2/mg/kg.

Clinical scores started on day 16 and continued daily. Mice were sacrificed on day 26. Clinical scores of mouse paws were based on a 0-5 scoring system with 0 being normal and 5 being the most severe edema and erythema conditions. ELISAs on collected plasma samples were performed using commercial kits: IL-1β (R & D systems, Minneapolis, Minn.), anti-collagen II (Chondrex, Redmont, Wash.), and Rheumatoid Factors (IgG) (Alpha Diagnostic International, San Antonio, Tex.).

CA4 at 50, 33, and 0 (vehicle) mg/ml in osmotic pumps (0.5 ul/hr; equivalent to 25, 16 and 0 mg/kg/day) was administered subcutaneously to murine collagen-induced arthritis model usin ghte B10RIII strain 12 days after the 1^{st} immunization (day 0) of type II collagen. The 33 mg/ml dose by the pump produced a plasma concentration of CA4 of approximately 30 nM on day 3 and 7 as measured by LC/MS. Dexamethasone at 0.2 mg/kg was injected daily subcutaneously as a positive control.

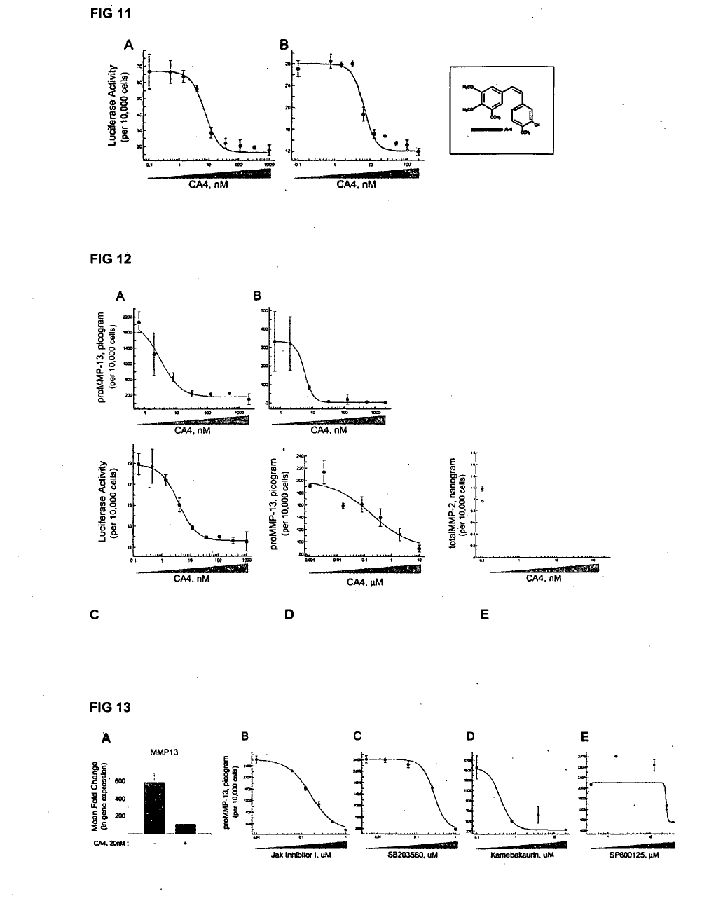

Eight of 15 mice in the 25 mg/kg/day group died by day 22. All of the mice in the 16 mg/kg/day group survived by day 22. However, skin lesions around the pump area were observed on day 23 for all the groups, including the vehicle control. CA4 dose-dependently reduced the arthritis incidence rate in the treated groups as opposed to the vehicle control between day 15 and 22 (FIG. 15A), and reduced the symptomatic macroscopic scores of the paws without dramatically affecting body weight (FIG. 15A-C)

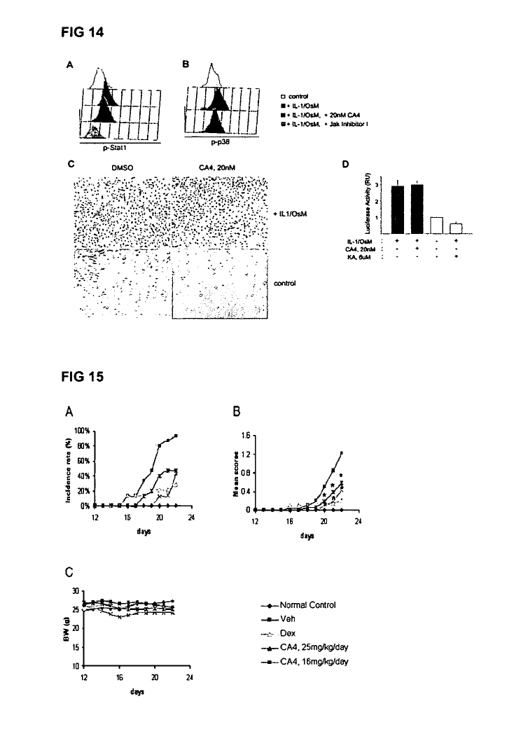

The protective effects of CA4 on the pathogenesis of arthritis were also evident histophathologically (FIG. 16). While mice in the vehicle group showed severe synovitis, massive infiltration of neutrophils and macrophages, nearly total loss of tibia and femoral cartilage and proteoglycan, fully developed panus, and marked bone resorption at their knee joints, mice in the 25 mg/kg/day group showed only minimal synovitis, minimal proteoglycan and cartilage loss, few infiltrated inflammatory cells, and no panus formation or bone resorption. This near complete joint protection was also seen in the dexamethasone-treated group and in the normal, unimmunized mice.

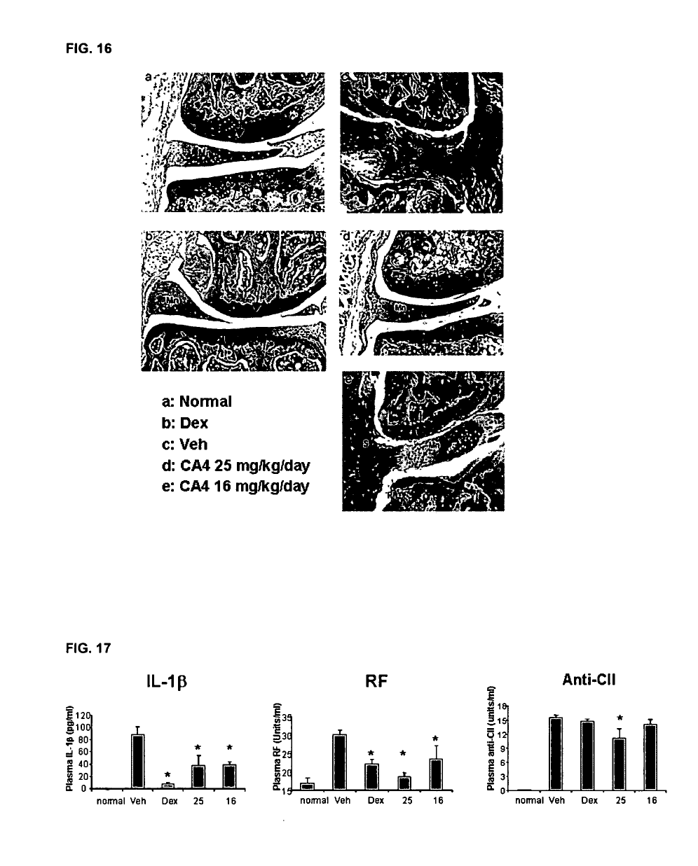

Mice in the 16 mg/kg/day group showed intermediate joint histopathological damages as opposed to the 25 mg/kg/day group and the vehicle control group. The serological markers of arthritis in these mice was also examined. As shown in FIG. 17, plasma IL-1β levels were reduced by more than 50% in both CA4-treated groups. Titers of Rheumatoid Factors, an indicator of arthritis, were improved significantly to lower levels in mice treated with both doses of CA4. Finally, the higher dose of CA4 significantly, albeit modestly, reduced the anti-type II collagen antibody titer, whereas dexamethasone failed to do so. Taken together, CA4 showed a decent amelioration of development of arthritis in this murine CIA model.

### Example 13

### Synthesis of Compounds Represent by (XIVc)

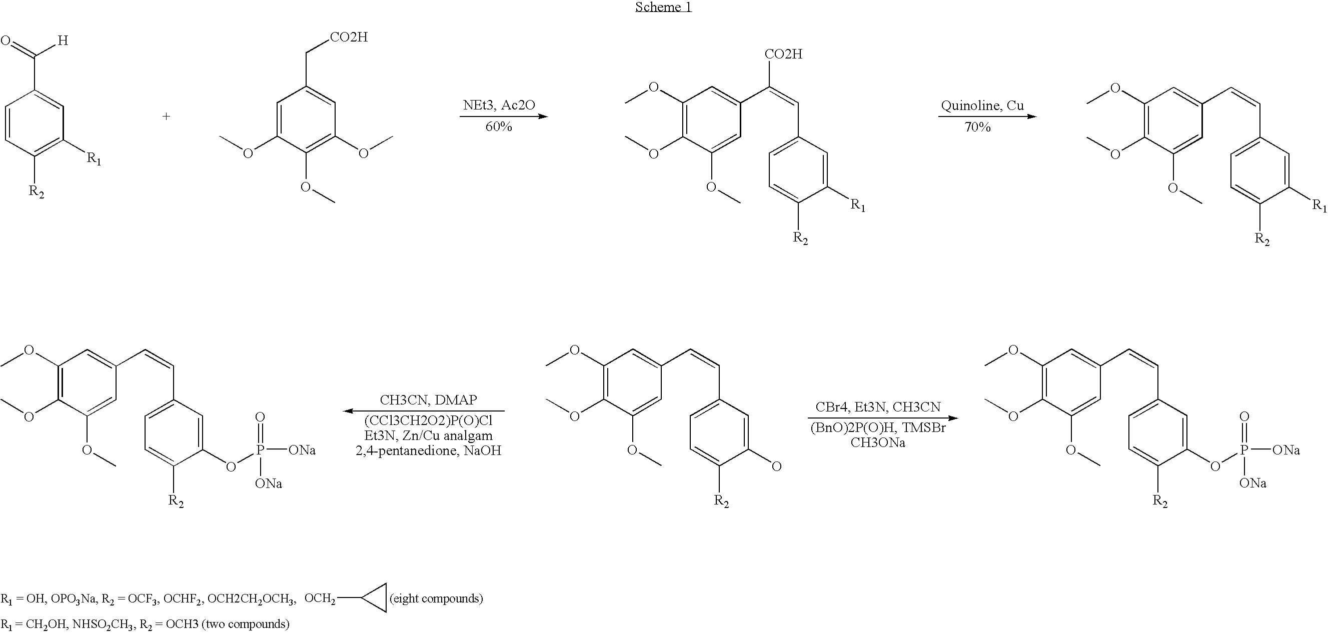

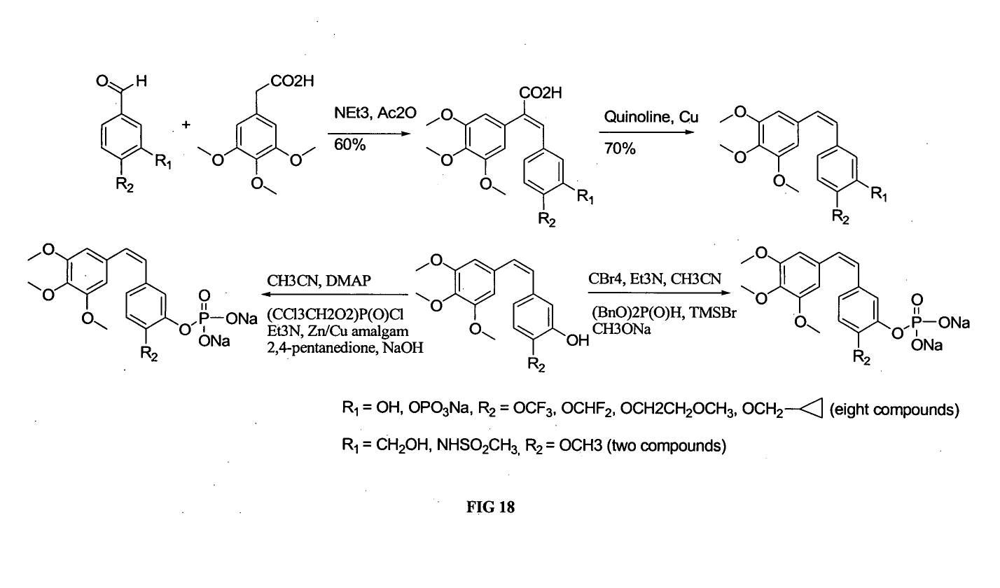

These compounds are synthesized by methods described in Gaukroger, et al. J. Org. Chem. 2001, 66, 8135, Letcher, et al. J. C. S. Perkin I. 1972, 206, Molho, et al. Bull.Soc.Chim.Fr. 1956, 78, Syedei, et al. U.S. Pat. No. 6,743,937, Syedei, et al. WO 02/006279 (the entire contents of each of which are incorporated herein by reference. (See FIG. 18).

While this invention has been particularly shown and described with references to preferred embodiments thereof, it will be understood by those skilled in the art that various changes in form and details may be made therein without departing from the scope of the invention encompassed by the appended claims.

The entire teachings of each cited reference are incorporated herein by reference.

What is claimed is:

1. A method of treating an inflammatory disorder in a subject in need of treatment thereof, comprising administering to the subject an effective amount of a compound represented by Structural Formula (I): 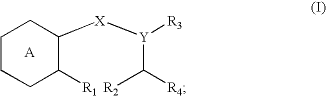
or a pharmaceutically acceptable salt or solvate thereof, wherein:
Ring A is optionally substituted, contains zero, one, two, or three double bonds, and is optionally fused to an aliphatic, aryl or heteroaryl ring;
X is an optionally substituted 1 to 3 carbon aliphatic chain that is optionally fused to a monocyclic, optionally substituted, aliphatic, heterocyclic, aryl, or heteroaryl ring, wherein one or two carbons in X are optionally replaced with —O—, —S—, or —NR^{e}—;
Y is carbon or nitrogen;
R₁ and R₂ are independently —H, —OH, —CN, —NO₂, —NR^{f}R^{e}, halogen, optionally substituted alkyl, or optionally substituted alkoxy; or R₁ and R₂ together link the carbons to which they are bonded with a bond, —O—, —S—, or —NR^{h}—;
R₃ and R⁴ are independently —H, —OH, —CN, —NO₂, —NRⁱR^{j}, halogen, optionally substituted alkyl, or optionally substituted alkoxy, or R⁴ is ═O; or R₃ and R⁴, taken together with the atoms to which they are bonded, form a monocyclic, optionally substituted, aliphatic, heterocyclic, aryl, or heteroaryl ring that is optionally fused to a monocyclic or bicyclic, optionally substituted, aliphatic, heterocyclic, aryl, or heteroaryl ring; and
the compound comprises at least one hydrogen atom bonded to an oxygen, nitrogen, or sulfur atom,
wherein R^{e}-R^{j} are independently —H or optionally substituted alkyl with the proviso that the compound is not combretastatin A4,
combretastatin A1, 1-(4-Methoxy-3-(5-nitrothien-2-yl) methoxy)phenyl-2-(3,4,5-trimethoxy)phenyl-Z-ethene, 1-(4-Methoxy-3-(1-(5-nitrothien-2-yl)ethoxy))phenyl-2-(3,4,5-trimethoxy)phenyl-Z-ethene, 1-(4-Methoxy-3-(5-nitrothien-2-yl)methoxycarbonyloxy)phenyl-2-(3,4,5-trimethoxy)phenyl-Z-ethene, 5-Methoxy-3-((3,4,4′,5-tetramethoxy-(Z)-stilbene-3′-yl)oxy)methyl-1,2-dimethylindole-4,7-dione, 3-((3,4,4′,5-Tetramethoxy-(Z)-stilbene-3′-yl)oxy)methyl-1,2-dimethyl-5-(4-methylpiperazin-1-yl)indole-4,7-dione, doxorubicin, daunorubicin, trimetrexate, methotrexate; etoposide, teniposide, topotecan, SN38; epothilone D, podophyllotoxin, vinblastine, vincristine, vinorelbine, paclitaxel, docetaxel, epirubicin, gefitinib, erlotinib, ZD6474, AZD2171, 1-(4-Methoxy-3-(2-(5-nitrothiophen-2-yl)propan-2-yl)oxyphenyl-2-(3,4,5-trimethoxy)phenyl-Z-ethene, 1-(4-Methoxy-3-(2-(4-nitrophenyl)propan-2-yl)oxyphenyl-2-(3,4,5-trimethoxy)phenyl-Z-ethene, 9-(7,8-Dihydroxy-2-methyl-hexahydro-pyrano[3,2-d][1,3]-dioxin-6-yloxy)-5-{3,5-dimethoxy-4-[1-methyl-1-(4-nitrophenyl)-ethoxy]-phenyl}-5,8,8a,9-tetrahydro-5aH-furo[3′, 4′:6,7]naphtho [2,3-d][1,3]dioxol-6-one, 6-(2-(4-nitrophenyl)propan-2-ylsulfanyl)-9H-purine, 1-(4-Methoxy-3-(1-methyl-4-(5-nitrothien-2-yl)piperidin-4-yl)oxycarbonyloxy)phenyl-2-(3,4,5-trimethoxy)phenyl-Z ethene, 1-(4-Methoxy-3-(2-(1-methyl-2-nitroimidazol-5-yl)propan-2-yl)oxyphenyl-2-(3,4,5-trimethoxy) phenyl-Z-ethene, 6-(2-(5-nitrothien-2-yl)propan-2-ylsulfanyl)-9H-purine, 1-(3-(1-Ethoxycarbonyl-1-(5-nitrothien-2-yl)ethoxy)-4-methoxy-phenyl)-2-(3,4,5-trimethoxyphenyl)-Z-ethene or N-(2-{3-[1-Methyl-1-(5-nitro-thiophen-2-yl)-ethoxy]-phenyl}-ethyl)-acetamide.

2. The method of claim 1, wherein the subject is human.

3. The method of claim 1, wherein the compound is selected from, fenbendazole, abietic acid, β-boswellic acid, mycophenolic acid, benzobromarone, colchicines, betulinic acid, 4-[[3,4-(Methylenedioxy)benzyl]amino]-6-chloroquinazoline (MBCQ), dienestrol, dicumarol, and pifithrine-α.

4. The method of claim 1, wherein the inflammatory disorder is rheumatoid arthritis.

5. The method of claim 1, wherein the compound has at least one substituent that is a carboxylic acid derivative or a bioisostere thereof.

6. The method of claim 5, wherein one or more substitutable atoms in the compound represented by Structural Formula (I) are substituted with a group independently selected from —F, —Cl, —Br, —I, —CN, —NO₂, —OR^{a}, —C(O)R^{a}, OC(O)R^{a}, —C(O)OR^{a}, SR^{a}, —C(S)R^{a}, —OC(S)R^{a}, —C(S)OR^{a}, —C(O)SR^{a}, —C(S)SR^{a}, S(O)R^{a}, —SO₂R^{a}, —SO₃R^{a}, —PO₂R^{a}R^{b}, —PO₃R^{a}R^{b}, —N(R^{a}R^{b}), C(O)N(R^{a}R^{b}), —C(O)NR^{a}N^{b}SO₂R^{c}, —C(O)NR^{a}SO₂R^{c}, —C(O)NR^{a}CN, —SO₂N(R^{a}R^{b}), SO₂N(R^{a}R^{b})—NR^{c}C(O)R^{a}, —NR^{c}C(O)OR^{a}, —NR^{c}C(O)N(R^{a}R^{b}), C(NR^{c})—N(R^{a}R^{b}), —NR^{d}—C(NR^{c})—N(R^{a}R^{b}), —NR^{a}N(R^{a}R^{b}), —CR^{c}═CR^{a}R, —C≡CR^{a}, ═O, ═S, ═CR^{a}R^{b}, ═NR^{a}, ═NOR^{a}, ═NNR^{a}, optionally substituted alkyl, optionally substituted cycloalkyl, optionally substituted aliphatic, optionally substituted cycloaliphatic, optionally substituted heterocyclic, optionally substituted benzyl, optionally substituted aryl, and optionally substituted heteroaryl; wherein R^{a}-R^{d} are each independently —H or optionally substituted aliphatic, optionally substituted cycloaliphatic, optionally substituted heterocyclic, optionally substituted benzyl, optionally substituted aryl, or optionally substituted heteroaryl, or —N(R^{a}R^{b}), taken together, is an optionally substituted heterocyclic group.

7. The method of claim 6, wherein the compound is represented by the following Structural Formula: 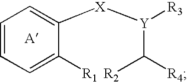
wherein Ring A′ is optionally substituted and is optionally fused to a monocyclic aliphatic, aryl or heteroaryl ring.

8. The method of claim 7, wherein the compound is represented by the following Structural Formula: 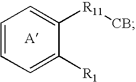
wherein R₁₁ is an optionally substituted C₃-C₁₂ aliphatic chain that is optionally interrupted by —O—, —S—, or —NR^{k}—; wherein R is —H or optionally substituted alkyl; and
CB is a carboxylic acid derivative or a bioisostere thereof.

9. The method of claim 8, wherein the compound is represented by the following Structural Formula: 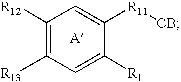
wherein R₁₂ and R₁₃ are independently —OR^{a}, —C(O)R^{a}, —OC(O)R^{a}, —C(O)OR^{a}, —N(R^{a}R^{b}), —C(O)N(R^{a}R^{b}), or optionally substituted alkyl, or, together with the carbons of Ring A′ to which they are bonded, form a 5 or 6 membered heterocycle; and
R₁₁ is an optionally substituted C₄-C₈ alkene.

10. The method of claim 9, wherein the compound is substituted at one or more substitutable positions with one or more substituents selected from —F, —Cl, —Br, —I, —CN, —NO₂, —OR^{a}, —OC(O)R^{a}, —C(O)OR^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}; —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, ═O, ═S, ═CR^{a}R^{b}, ═NR^{a}, ═NOR^{a}, ═NNR^{a}, C1-C4 alkyl, and C1-C4 haloalkyl.

11. The method of claim 10, wherein the compound is represented by the following Structural Formula: 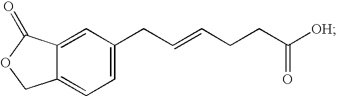
wherein the compound is substituted at one or more substitutable positions with one or more substituents selected from —F, —Cl, —Br, —I, —CN, —NO₂, —OR^{a}, C(O)OR^{a}, —N(R^{a}R^{b}), —C(O)N(R^{a}R^{b}), —NR^{c}C(O)R^{a}, —NR^{c}C(O)OR^{a}, —CR^{c}═CR^{a}R^{b}, ═O, ═S, ═CR^{a}R^{b}, ═NR^{a}, ═NOR^{a}, ═NNR^{a}, C1-C4 alkyl, and C1-C4 haloalkyl.

12. The method of claim 11, wherein the compound is:

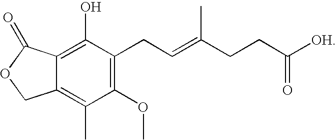

13. The method of claim 7, wherein the compound is represented by the following Structural Formula: 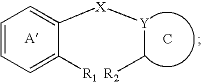
wherein Ring C is an optionally substituted 5 to 12 membered, monocyclic or bicyclic, aliphatic, heterocyclic, aryl, or heteroaryl ring.

14. The method of claim 13, wherein the compound is represented by the following Structural Formula: 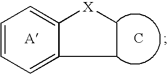
wherein
Ring C has 2 or 3-double bonds;
one of X and Ring A′ is substituted with a carboxylic acid derivative or a bioisostere thereof; and
the compound is substituted at one or more substitutable positions with one or more substituents selected from —F, —Cl, —Br, —I, —CN, —NO₂, —OR^{a}, —OC(O)R^{a}, —C(O)OR^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, ═O, ═S, ═CR^{a}R^{b}, ═NR^{a}, ═NOR^{a}, ═NNR^{a}, and optionally substituted alkyl.

15. The method of claim 14, wherein the compound is substituted at two or more substitutable positions with one or more substituents selected from —F, —Cl, —Br, —I, —CN, —NO₂, —OR^{a}, —C(O)OR^{a}, —N(R^{a}R^{b}), —C(O)N(R^{a}R^{b}), —NR^{c}C(O)R^{a}, —NR^{c}C(O)OR^{a}, —CR^{c}═CR^{a}R^{b}, ═O, ═S, ═CR^{a}R^{b}, ═NR^{a}, ═NOR^{a}, ═NNR^{a}, C1-C4 alkyl, and C1-C4 haloalkyl.

16. The method of claim 15 wherein the compound is represented by the following Structural Formula: 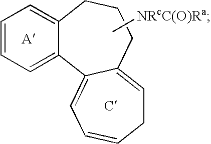
wherein the compound is substituted at two or more substitutable positions with one or more substituents selected from —Cl, —Br, —R^{k}, —OR^{k}, —C(O)OR^{k}, —NHC(O)R^{k}, —NHC(O)OR^{k}, —C(═CH2)R^{k}, ═O, ═CHR^{k}, and ═NR^{k}, wherein R^{k} is methyl, ethyl, propyl, 2-propyl, butyl, sec-butyl, or tertiary butyl.

17. The method of claim 16, wherein the compound is:

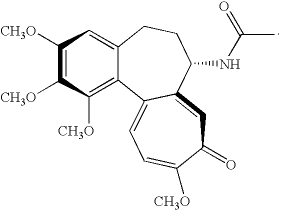

18. The method of claim 13 wherein the compound is represented by the following Structural Formula: 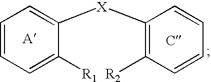
wherein
Ring C″ is optionally substituted and is optionally fused to an aliphatic, aryl or heteroaryl ring;
at least one of Ring A′ and Ring C″ is substituted with a carboxylic acid derivative or a bioisostere thereof; and
the compound is substituted at one or more substitutable positions with one or more substituents selected from —F, —Cl, —Br, —I, —CN, —NO₂, —OR, —OC(O)R^{a}, —C(O)OR^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), C(O)NR^{a}N(R^{a}R^{b}), —SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —C(NR^{c})—N(R^{a}R^{b}), NR^{d}—C(NR^{c})—N(R^{a}R^{b}), NR^{a}N(R^{a}R^{b}), —CR^{c}═CR^{a}R^{b}, ═O, ═S, ═CR^{a}R^{b}, ═NR^{a}, ═NOR^{a}, ═NNR^{a}, C1-C4 alkyl, and C1-C4 haloalkyl.

19. The method of claim 18 wherein the compound is represented by the following Structural Formula: 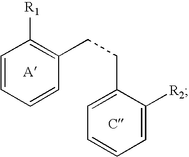
wherein
R₁ and R₂ are independently —H, —OH, —CN, —NO₂, —NR^{f}R^{g}, halogen, optionally substituted alkyl, or optionally substituted alkoxy;
the compound is substituted at two or more substitutable positions with one or more substituents selected from —F, —Cl, —Br, —I, —CN, —NO₂, —OR^{a}, —C(O)OR^{a}, —N(R^{a}R^{b}), —C(O)N(R^{a}R^{b}), —NR^{c}C(O)R^{a}, —NR^{c}C(O)OR^{a}, —CR^{c}═CR^{a}R^{b}, ═O, ═S, ═CR^{a}R^{b}, ═NR^{a}, ═NOR^{a}, ═NNR^{a}, C1-C4 alkyl, and C1-C4 haloalkyl; and
- - - is a single or double bond.

20. The method of claim 19, wherein the compound is represented by the following Structural Formula: 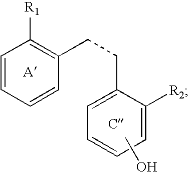
wherein the compound is substituted at one or more substitutable positions with one or more substituents selected from —Cl, —Br, —R^{k}, —OR^{k}, —C(O)OR^{k}, —NHC(O)R^{k}, —NHC(O)OR^{k}, —C(═CH2)R^{k}, ═O, ═CHR^{k}, and ═NR^{k}; wherein R^{k} is methyl, ethyl, propyl, 2-propyl, butyl, sec-butyl, or tertiary butyl.

21. The method of claim 20, wherein the compound is represented by the following Structural Formula:

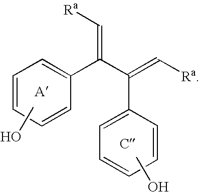

22. The method of claim 21, wherein the compound is:

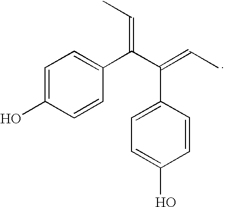

23. The method of claim 20, wherein the compound is represented by the following Structural Formula:

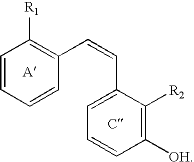

24. The method of claim 13, wherein the compound is represented by the following Structural Formula: 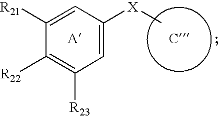
wherein
Ring C′″ is an optionally substituted, 5-12 membered, monocyclic or bicyclic, heteroaryl or heterocyclic ring;
R₂₁, R₂₂, and R₂₃ are independently —H, —OH, —F, —Cl, —Br, or alkoxy; or R₂₁ and R₂₂ together are a methylene dioxy or ethylene dioxy group forming a 5 or 6 member ring fused to the aryl ring to which they are bonded; and
the compound is substituted at one or more substitutable positions with one or more substituents selected from —F, —Cl, —Br, —I, —CN, —NO₂, —OR^{a}, —OC(O)R^{a}, —C(O)OR^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R), —NR^{c}C(O)R^{a}, —NR^{c}C(O)OR^{a}, —C(NR^{c})N(R^{a}R^{b}), —NR^{d}—C(NR^{c})—N(R^{a}R^{b}), —NR^{a}N(R^{a}R^{b}), —CR^{c}═CR^{a}R^{b}, ═O, ═S, ═CR^{a}R^{b}, ═NR^{a}, ═NOR^{a}, ═NNR^{a}, C1-C4 alkyl, and C1-C4 haloalkyl.

25. The method of claim 24, wherein the compound is represented by the following Structural Formula: 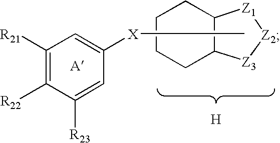
wherein
Z₁, Z₂, and Z₃ are each independently C, N, S, or O, provided that at least one of Z₁, Z₂, and Z₃ is N, S, or O, and at least one is C;
optionally substituted bicyclic ring H is saturated or unsaturated; and
the compound is substituted at one or more substitutable positions with one or more substituents selected from —F, —Cl, —Br, —I, —CN, —NO₂, —OR^{a}, —C(O)OR^{a}, —N(R^{a}R^{b}), —C(O)N(R^{a}R^{b}), —NR^{c}C(O)R^{a}, —NR^{c}C(O)OR^{a}, —CR^{c}═CR^{a}R^{b}, ═O, ═S, ═CR^{a}R^{b}, ═NR^{a}, ═NOR^{a}, ═NNR^{a}, C1-C4 alkyl, and C1-C4 halo alkyl.

26. The method of claim 25 wherein Z₁ and Z₃ are independently N, S, or O.

27. The method of claim 26 wherein the compound is represented by the following Structural Formula: 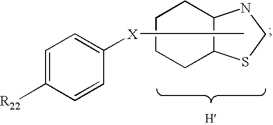
wherein ring H′ is unsaturated and is substituted with at least one substituent selected from —F, —Cl, —Br, —I, —CN, —NO₂, —OR^{a}, C(O)OR^{a}, —N(R^{a}R^{b}), —C(O)N(R^{a}R^{b}), —NR^{c}C(O)R^{a}, NR^{c}C(O)OR^{a}, —CR^{c}═CR^{a}R^{b}, ═O, ═S, ═CR^{a}R^{b}, ═NR^{a}, ═NOR^{a}, ═NNR^{a}, C1-C4 alkyl, and C1-C4 haloalkyl.

28. The method of claim 27 wherein X is C1-C3 alkyl optionally substituted with ═O.

29. The method of claim 28 wherein the compound is:

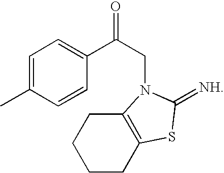

30. The method of claim 25, wherein the compound is represented by the following Structural Formula: 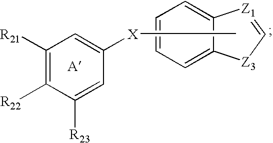
wherein
the compound is substituted at one or more substitutable positions with one or more substituents selected from Cl, —Br, —R^{k}, —OR^{k}, —C(O)OR^{k}, —NHC(O)R^{k}, —NHC(O)OR^{k}, —C(═CH2)R^{k}, ═O, ═CHR^{k}, and ═NR^{k}, wherein R^{k} is methyl, ethyl, propyl, 2-propyl, butyl, sec-butyl, and tertiary butyl.

31. The method of claim 30, wherein Z₁ and Z₃ are N.

32. The method of claim 31, wherein X is —O— or —S—.

33. The method of claim 32 wherein the compound is:

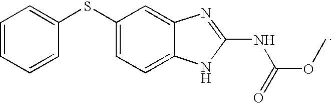

34. The method of claim 25, wherein Z₁ is N or C, Z₃ is S or O, and R₂₂ is —OH.

35. The method of claim 34, wherein X is C1-C3 alkyl optionally substituted with ═O.

36. The method of claim 35, wherein the compound is:

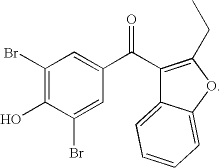

37. The method of claim 24, wherein Ring C′″ is an optionally substituted, 10 membered, bicyclic heteroaryl group.

38. The method of claim 37, wherein X is optionally substituted —CH₂CH₂—, —CH═CH—, —CH₂NH—, or —NHCH₂—.

39. The method of claim 38, wherein the compound is represented by the following Structural Formula: 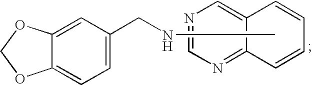
wherein the compound is substituted at one or more substitutable positions with one or more substituents selected from Cl, —Br, —R^{k}, OR^{k}, —C(O)OR^{k}, —NHC(O)R^{k}, —NHC(O)OR^{k}, —C(═CH2)R^{k}, ═O, ═CHR^{k}, and ═NR^{k}, wherein R^{k} is methyl, ethyl, propyl, 2-propyl, butyl, sec-butyl, or tertiary butyl.

40. The method of claim 39, wherein the compound is:

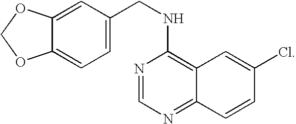

41. The method of claim 7, wherein the compound is represented by the following Structural Formula: 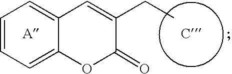
wherein
Ring C′″ is an optionally substituted, 5-12 membered, monocyclic or bicyclic, heteroaryl or heterocyclic ring; and
the compound is substituted at one or more substitutable positions with one or more substituents selected from —F, —Cl, —Br, —I, —CN, —NO₂, —OR^{a}, C(O)OR^{a}, N(R^{a}R^{b}), —C(O)N(R^{a}R), —NR^{c}C(O)R^{a}, —NR^{c}C(O)OR^{a}, —CR^{c}═CR^{a}R^{b}, ═O, ═S, ═CR^{a}R^{b}, ═NR^{a}, ═NOR^{a}, NNR^{a}, C1-C4 alkyl, and C1-C4 haloalkyl.

42. The method of claim 41 wherein the compound is:

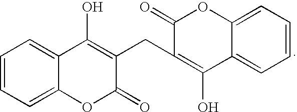

43. The method of claim 6, wherein the compound is represented by the following Structural Formula: 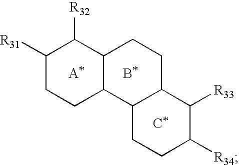
wherein:
R₃₁ is —H or alkyl and R₃₂ is alkyl; or R₃₁ and R₃₂ together form an optionally substituted 5 or 6 membered aliphatic or heterocyclic ring that is optionally fused to a 5 or 6 membered aliphatic or heterocyclic ring;
R₃₃ is —H or alkyl and R₃₄ is alkyl; or R₃₃ and R₃₄ together with the atoms of Ring C* to which they are bonded, form an optionally substituted, 5 or 6 membered, aliphatic or heterocyclic ring that is optionally fused to a 5 to 12 membered, aliphatic or heterocyclic, monocyclic or bicyclic ring; and
Rings A*, B*, and C* contain zero, one, or two double bonds, wherein the compound is substituted at one or more substitutable positions with one or more substituents selected from —F, —Cl, —Br, —I, —CN, —NO₂, —OR^{a}, —OC(O)R^{a}, —C(O)OR^{a}, —SO₂R^{a}, —SO₃R^{a}, PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, SO₂N(R^{a}R^{b}), —SO₂N(R^{a}R^{b}), —NR^{c}C(O)R^{a}, —NR^{c}C(O)OR^{a}, —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, ═O, ═S, ═CR^{a}R^{b}, ═NR^{a}, ═NOR^{a}, ═NNR^{a}, C1-C4 alkyl, and C1-C4 haloalkyl.

44. The method of claim 43, wherein Ring A* has at least one substituent that is a carboxylic acid derivative or a bioisostere thereof.

45. The method of claim 44, wherein the compound is represented by the following Structural Formula: 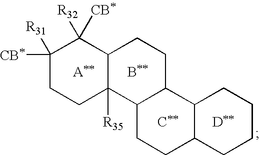
wherein:
one CB* is —H and one is a carboxylate derivative or bioisostere thereof; R₃₅ is alkyl;
optionally substituted Ring D** is optionally fused to a substituted or unsubstituted five or six membered aliphatic ring; and
Rings A**, B**, C** and D** each have zero, one, or two double bonds,
wherein the compound is substituted at two or more substitutable positions with one or more substituents selected from —F, —Cl, —Br, —I, —CN, —NO₂, —OR^{a}, C(O)OR^{a}, N(R^{a}R^{b}), C(O)N(R^{a}R^{b}) —NR^{c}C(O)R^{a}, —NR^{c}C(O)OR^{a}, —CR^{c}═CR^{a}R^{b}, ═O, ═S, ═CR^{a}R^{b}, ═NR^{a}, ═NOR^{a}, ═NNR^{a}, C₁-C₄ alkyl, and C₁-C₄ haloalkyl.

46. The method of claim 45, wherein the compound is represented by the following Structural Formula: 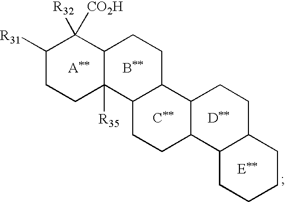
wherein
the compound is substituted at one or more substitutable positions with one or more substituents selected from —Cl, —Br, —R^{k}, —OR^{k}, —C(O)OR^{k}, —NHC(O)R^{k}, —NHC(O)OR^{k}, —C(═CH2)R^{k}, ═O, ═CHR^{k}, and ═NR^{k}, wherein R^{k} is methyl, ethyl, propyl, 2-propyl, butyl, sec-butyl, or tertiary butyl; and
Ring E** is optionally substituted and has zero, one, or two double bonds.

47. The method of claim 46, wherein the compound is:

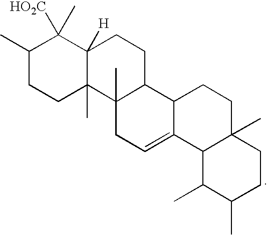

48. The method of claim 45, wherein the compound is represented by the following Structural Formula: 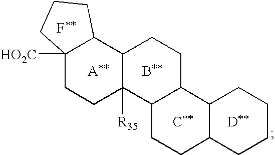
wherein:
the compound is substituted at one or more substitutable positions with one or more substituents selected from —Cl, —Br, —R^{k}, —OR^{k}, —C(O)OR^{k}, —NHC(O)R^{k}, —NHC(O)OR^{k}, —C(═CH2)R^{k}, ═O, CHR^{k}, and ═NR^{k}, wherein R^{k} is methyl, ethyl, propyl, 2-propyl, butyl, sec-butyl, or tertiary butyl; and
Ring F** is optionally substituted and has zero, one, or 2 double bonds.

49. The method of claim 48, wherein the compound is:

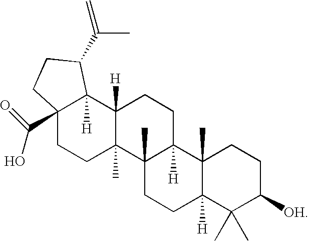

50. The method of claim 43, wherein the compound is represented by the following Structural Formula: 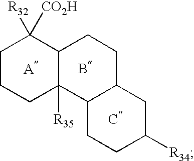
wherein the compound is substituted at one or more substitutable positions with one or more substituents selected from —Cl, —Br, —R^{k}, —OR^{k}, —C(O)OR^{k}, —NHC(O)R^{k}, —NHC(O)OR^{k}, —C(═CH2)R^{k}, ═O, ═CHR^{k}, and ═NR^{k}, wherein R^{k} is methyl, ethyl, propyl, 2-propyl, butyl, sec-butyl, or tertiary butyl.

51. The method of claim 50, wherein the compound is:

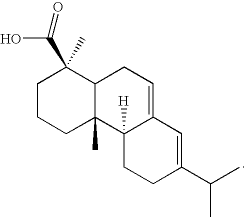

52. The method of claim 1, wherein the inflammatory disorder is osteoarthritis.

53. A method of inhibiting oncostatin M signaling in a subject in need of such inhibition, comprising administering an effective amount of a compound represented by Structural Formula (I): 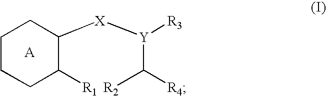
or a pharmaceutically acceptable salt or solvate thereof, wherein:
Ring A is optionally substituted, contains zero, one, two, or three double bonds, and is optionally fused to an aliphatic, aryl or heteroaryl ring;
X is an optionally substituted 1 to 3 carbon aliphatic chain that is optionally fused to a monocyclic, optionally substituted, aliphatic, heterocyclic, aryl, or heteroaryl ring, wherein one or two carbons in X are optionally replaced with —O—, —S—, or —NR^{e}—;
Y is carbon or nitrogen;
R₁ and R₂ are independently —H, —OH, —CN, —NO₂, —NR^{f}R^{g}, halogen, optionally substituted alkyl, or optionally substituted alkoxy; or R₁ and R₂ together link the carbons to which they are bonded with a bond, —O—, —S—, or —NR^{h}—;
R₃ and R⁴ are independently —H, —OH, —CN, —NO₂, —NRⁱR^{j}, halogen, optionally substituted alkyl, or optionally substituted alkoxy, or R⁴ is ═O; or R₃ and R⁴, taken together with the atoms to which they are bonded, form a monocyclic, optionally substituted, aliphatic, heterocyclic, aryl, or heteroaryl ring that is optionally fused to a monocyclic or bicyclic, optionally substituted, aliphatic, heterocyclic, aryl, or heteroaryl ring; and
the compound comprises at least one hydrogen atom bonded to an oxygen, nitrogen, or sulfur atom,
wherein R^{e}-R^{j} are independently —H or optionally substituted alkyl.

54. A method of inhibiting MMP-13 expression in a subject in need of such inhibition, comprising administering an effective amount of a compound represented by Structural Formula (I): 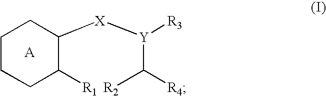
or a pharmaceutically acceptable salt or solvate thereof, wherein:
Ring A is optionally substituted, contains zero, one, two, or three double bonds, and is optionally fused to an aliphatic, aryl or heteroaryl ring;
X is an optionally substituted 1 to 3 carbon aliphatic chain and Y is carbon or nitrogen; or, Y is carbon and one or two carbons in X are optionally replaced with —O—, —S—, or —NR^{e}—;
R₁ and R₂ are independently —H, —OH, —CN, —NO₂, —NR^{f}R^{g}, halogen, optionally substituted alkyl, or optionally substituted alkoxy; or R₁ and R₂ together link the carbons to which they are bonded with a bond, —O—, —S—, or —NR^{e}—;
R₃ and R⁴ are independently —H, —OH, —CN, —NO₂, —NR^{f}R^{g}, halogen, optionally substituted alkyl, or optionally substituted alkoxy, or R⁴ is ═O; or R₃ and R⁴, taken together with the atoms to which they are bonded, form a monocyclic, optionally substituted, aliphatic, heterocyclic, aryl, or heteroaryl ring that is optionally fused to a monocyclic or bicyclic, optionally substituted, aliphatic, heterocyclic, aryl, or heteroaryl ring; and
the compound comprises at least one hydrogen atom bonded to an oxygen, nitrogen, or sulfur atom,
wherein R^{e}-R^{j} are independently —H or optionally substituted alkyl.

55. The method of claim 1, wherein the subject suffers from synovitis, proteoglycan loss, cartilage loss, panus formation or bone resorption.

56. A method of treating osteoarthritis in a subject in need of treatment thereof, comprising administering to the subject an effective amount of a compound represented by Structural Formula (I): 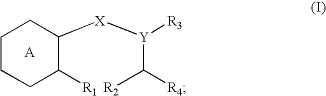
or a pharmaceutically acceptable salt or solvate thereof, wherein:
Ring A is optionally substituted, contains zero, one, two, or three double bonds, and is optionally fused to an aliphatic, aryl or heteroaryl ring;
X is an optionally substituted 1 to 3 carbon aliphatic chain that is optionally fused to a monocyclic, optionally substituted, aliphatic, heterocyclic, aryl, or heteroaryl ring, wherein one or two carbons in X are optionally replaced with —O—, —S—, or —NR^{e}—;
Y is carbon or nitrogen;
R₁ and R₂ are independently —H, —OH, —CN, —NO₂, —NR^{f}R^{g}, halogen, optionally substituted alkyl, or optionally substituted alkoxy; or R₁ and R₂ together link the carbons to which they are bonded with a bond, —O—, —S—, or —NR^{h}—;
R₃ and R⁴ are independently —H, —OH, —CN, —NO₂, —NRⁱR^{j}, halogen, optionally substituted alkyl, or optionally substituted alkoxy, or R⁴ is ═O; or R₃ and R⁴, taken together with the atoms to which they are bonded, form a monocyclic, optionally substituted, aliphatic, heterocyclic, aryl, or heteroaryl ring that is optionally fused to a monocyclic or bicyclic, optionally substituted, aliphatic, heterocyclic, aryl, or heteroaryl ring; and
the compound comprises at least one hydrogen atom bonded to an oxygen, nitrogen, or sulfur atom,
wherein R^{e}-R^{j} are independently —H or optionally substituted alkyl.

57. A method of treating an inflammatory disorder in a subject in need of treatment thereof, comprising administering to the subject an effective amount of a compound represented by Structural Formula (I): 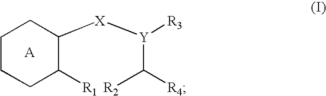
or a pharmaceutically acceptable salt or solvate thereof, wherein:
Ring A is optionally substituted, contains zero, one, two, or three double bonds, and is optionally fused to an aliphatic, aryl or heteroaryl ring;
X is an optionally substituted 1 to 3 carbon aliphatic chain that is optionally fused to a monocyclic, optionally substituted, aliphatic, heterocyclic, aryl, or heteroaryl ring, wherein one or two carbons in X are optionally replaced with —O—, —S—, or —NR^{e}—;
Y is carbon or nitrogen;
R₁ and R₂ are independently —H, —OH, —CN, —NO₂, —NR^{f}R^{g}, halogen, optionally substituted alkyl, or optionally substituted alkoxy; or
R₁ and R₂ together link the carbons to which they are bonded with a bond, —O—, —S—, or —NR^{h}—;
R₃ and R⁴ are independently —H, —OH, —CN, —NO₂, —NRⁱR^{j}, halogen, optionally substituted alkyl, or optionally substituted alkoxy, or R⁴ is ═O; or R₃ and R⁴, taken together with the atoms to which they are bonded, form a monocyclic, optionally substituted, aliphatic, heterocyclic, aryl, or heteroaryl ring that is optionally fused to a monocyclic or bicyclic, optionally substituted, aliphatic, heterocyclic, aryl, or heteroaryl ring; and
the compound comprises at least one hydrogen atom bonded to an oxygen, nitrogen, or sulfur atom,
wherein R^{e}-R^{j} are independently —H or optionally substituted alkyl with the proviso that the disease is not rheumatoid arthritis.

58. A method of treating an inflammatory disorder in a subject in need thereof, comprising administering to the subject an effective amount of a compound represented by the following structural formula: 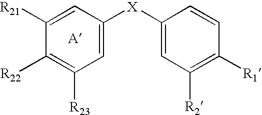
wherein:
R₁′ and R₂′ are each independently —F, —Cl, —Br, —I, —CN, —NO₂, —OR^{a}, —OC(O)R^{a}, —C(O)OR^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, ═O, ═S, ═CR^{a}R^{b}, ═NR^{a}, ═NOR^{a}, ═NNR^{a}, —OPO₃R^{x}, —NR^{a}SO₂R^{c} or optionally substituted alkyl; R₂₁, R₂₂, and R₂₃ are independently —H, —OH, —F, —Cl, —Br, or alkoxy; or R₂₁ and R₂₂ together are a methylene dioxy or ethylene dioxy group forming an optionally substituted 5 or 6 member ring fused to the aryl ring to which they are bonded;
R^{a}-R^{d} are each independently —H or an optionally substituted aliphatic, optionally substituted cycloaliphatic, optionally substituted heterocyclic, optionally substituted benzyl, optionally substituted aryl, or optionally substituted heteroaryl, or, —N(R^{a}R^{b}), taken together, is an optionally substituted heterocyclic group; and
each R^{x} is independently halo, —H, an optionally substituted aliphatic, optionally substituted cycloaliphatic, optionally substituted heterocyclic, optionally substituted benzyl, optionally substituted aryl, or optionally substituted heteroaryl, or, —N(R^{a}R^{b}), taken together, is an optionally substituted heterocyclic group.

59. The method of claim 58 wherein the compound is represented by the following structural formula: 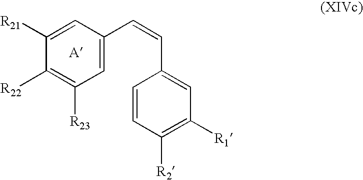
wherein:
R₁′ and R₂′ are each independently OR^{a}, —OPO₃R^{x}, —NR^{a}SO₂R^{c} or optionally substituted alkyl.

60. The method of claim 59 wherein:
R₂₁-R₂₃ are —OCH₃;
R₁′ is —OH or —OPO₃Na; and
R₂′ is OCF₃, —OCHF₂, —OCH₂CH₂OCH₃ or —OCH₂(C3 cycloalkyl).

61. The method of claim 60 wherein:
R₂₁-R₂₃ are —OCH₃;
R₁′ is —CH₂OH or —NHSO₂CH₃; and
R₂′ is OCH₃.

62. A compound represented by the following structural formula: 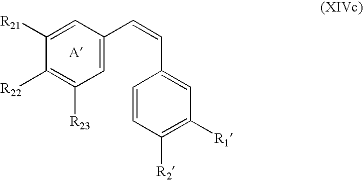
wherein
R₁′ and R₂′ are each independently OR^{a}, —OPO₃R^{x}, —NR^{a}SO₂R^{c} or optionally substituted alkyl; and
R₂₁, R₂₂, and R₂₃ are independently —H, —OH, —F, —Cl, —Br, or alkoxy; or R₂₁ and R₂₂ together are a methylene dioxy or ethylene dioxy group forming an optionally substituted 5 or 6 member ring fused to the aryl ring to which they are bonded.

63. The compound of claim 62 wherein:
R₂₁-R₂₃ are —OCH₃;
R₁′ is —OH or —OPO₃Na; and
R₂′ is OCF₃, —OCHF₂, —OCH₂CH₂OCH₃ or —OCH₂(C3 cycloalkyl).

64. The compound of claim 62 wherein:
R₂₁-R₂₃ are —OCH₃;
R₁′ is —CH₂OH or —NHSO₂CH₃; and
R₂′ is OCH₃.
